(19) United States
(12) Patent Application Publication
(10) Pub. No.: US 2010/0233701 A1
(43) Pub. Date: Sep. 16, 2010

(54) Use of non-clonal chromosomal aberrations for cancer research and clinical diagnosis

(76) Inventors: Henry H. Heng, Riverview, MI (US); Christine Ye a/k/a Jing Ye, Windsor (CA)

Correspondence Address: Rohm & Monsanto, PLC, 12 Rathbone Place, Grosse Pointe, MI, 48230, US

(21) Appl. No.: 12/655,368

(22) Filed: Dec. 28, 2009

Related U.S. Application Data
(63) Continuation-in-part of application No. 12/583,194, filed on Aug. 14, 2009.
(60) Provisional application No. 61/188,916, filed on Aug. 14, 2008.

(51) Int. Cl. C12Q 1/68

(52) U.S. Cl. 435/6

(57) ABSTRACT

A diagnostic method of determining tumorigenicity of a tissue specimen includes the steps of determining the magnitude of genome diversity in the tissue specimen, and diagnosing a likelihood of cancer in response thereto. The magnitude of genome diversity includes the determination of karyotypic heterogeneity in tissue specimen, illustratively by detecting non-clonal chromosome aberrations (NCCAs). The detection of NCCAs includes the identification of various types and frequency of NCCAs, and diagnosis is responsive to the step of detecting the frequency of NCCAs. Detection of NCCAs includes the further step of screening lymphocytes. Also, the step of determining the presence of elevated genome diversity includes the step of applying Spectral Karyotyping to detect structural and numerical aberrations throughout the genome. The diagnostic method is useful to determine drug resistance in a patient and potential harmfulness, to evaluate the side effects of drugs, and to measure genome system stress.

### RELATIONSHIP TO OTHER APPLICATION(S)

This application is a continuation-in-part patent application of U.S. Ser. No. 12/583,194, filed Aug. 14, 2009, which claims the benefit of the filing date of Provisional Patent Application Ser. No. 61/188,916, filed on Aug. 14, 2008, the disclosures of which are incorporated herein by reference.

### GOVERNMENT RIGHTS

This invention was made in part under contract awarded by National Cancer Institute—National Institute of Health, Contract Number R01-CA100247. The government may have certain rights in the invention.

### BACKGROUND OF THE INVENTION

1. Field of the Invention

This invention relates generally to diagnostic or evaluation methods of detecting the characteristics of cancer or and other complex diseases, and more particularly, to a diagnostic methodology for determining the likelihood of the presence of cancer, or of developing cancer, or of monitoring chemotherapy, or evaluating effectiveness or side effects of drugs, in response to genomic or system instability, caused by disease conditions, or induced by various types of system stress.

2. Description of the Related Art

Increasing attention is focusing on chromosomal and genome structure in cancer research due to the fact that genomic instability plays a principal role in cancer initiation, progression and response to chemotherapeutic agents. The integrity of the genome (including structural, behavioral and functional aspects) of normal and cancer cells can be monitored with direct visualization by using a variety of cutting edge molecular cytogenetic technologies that are now available in the field of cancer research.

Cytogenetic visualization technologies have traditionally played an important role in cancer research. Both the chromosomal number changes or aneuploidy, and the telomeric deficient mediated chromosomal breakage-fusion-bridge cycle has long been linked to the cancer phenotype and chromosomal instability. Many chromosomal aberrations, particularly translocations or inversions are closely associated with a specific morphological or phenotypic subtype of leukemia, lymphoma or sarcoma. As a result, chromosomal analysis of patient samples has become an essential component of cancer research.

The identification of a chromosome translocation involving a reciprocal rearrangement of chromosomes 8 and 21 in patients with a form of acute myelogenous leukemia (AML), and the identification of a translocation involving chromosomes 9 and 22 in patients with chronic myeloid leukemia (CML), has initiated more than 100 genes cloned from translocation breakpoints. In the past three decades, over 30,000 cancer cases have been analyzed by chromosomal karyotyping employing one basic visualization method using normal chromosomes as a standard, to search for the correlation between a specific karyotype and a specific type of cancer, which revealed more than 600 acquired, recurrent, balanced chromosome rearrangements. Among these analyses, a great deal of attention has been focused on clonal chromosomal changes in the identification of both primary and secondary abnormalities. These clonal abnormalities, particularly if complex, are significant to neoplasia. As a result, these chromosomal visualization methods have served as an important tool for both cancer research and diagnosis. However, only clonal chromosomal aberrations (CCAs) have been used in diagnosis.

In recent years, extensive research has been performed with molecular probes targeting specific regions of the genome for detecting gene deletions and amplifications and copy number variations. With the development of live images as well as the maturation of FISH related technologies, more direct visualization approaches are available to cancer biology.

With the completion of the sequencing phase of both human and mouse genome projects, one of the next priorities is the systematic study of genomic structure relative to function as well as abnormalities associated with the cancer phenotype based on recent emerging genomic information. A powerful application of newly available technology is the use of microarrays to correlate specific genes or pathways to types or stages of cancer, particularly when used in conjunction with the tissue micro-dissection method. The challenge for this approach is the complexity of the karyotypical changes that occur and the karyotypical heterogeneity that is associated with cancer cell lines and tumor samples. It is therefore necessary to carefully karyotype the cell lines/tissues being studied before microarray analysis is performed.

CGH technology has been effective in establishing possible karyotype patterns by pinpointing the gains or loses within specific chromosomal regions. Since CGH data focuses on clonal karyotype changes it could miss non-clonal changes that occur at an early stage of cancer development (this is further discussed in the following section). Similarly, the heterogeneity of tumor samples makes data interpretation difficult with significant exceptions occurring when various samples of the same tumors were analyzed.

Gene knock out technology has produced a large collection of mouse models that can be used to study genomic aberrations that occur during cancer development. Particularly with the recent development of RNA interference technology, the correlation between different pathways defined by key genes and the genomic structure/function can be analyzed in great detail. The technical challenge for genome structure and function studies by using this approach is to develop a system that can monitor the genome structure and changes caused by these targeted genes. It would be ideal to directly visualize the changes occurring before and after dysfunction of genes that are expected to be involved in the maintenance of the integrity of the genome. Such a direct visualization system will fill the gaps between molecular biology and cytology, between studies using in vitro and in vivo assays, and could be used for comparative analysis between normal and cancer conditions.

One re-emerging concept in cancer research is that epigenetic events also play an important role in the evolution of cancer. Cancer often displays aberrant methylation of promoter regions, which is linked to the loss of gene function. Such heritable DNA changes are not mediated by altered nucleotide sequences and might involve the formation of transcriptionally repressive chromatin. Visualization methods are urgently needed to study cancer related epigenetic phenomena.

Traditional strategies in cancer research have been focused on the identification and characterization of the general patterns of genetic aberrations and in particular, key “cancer gene” mutations. The underlying principle has been specific types of cancer are caused by sequential genetic events occurring during “cancer development.” This gene-centric view has dominated the field of cancer research for decades resulting in the concentration of research effort on defining mutated oncogenes, tumor suppressor genes and their molecular pathways.

Despite the initial success of identifying a number of gene mutations that had a high penetration among certain patient populations, most subsequently identified gene mutations have displayed low frequencies among patients. Further, the list of cancer genes continues to grow, which brings in to question the goals and rationale of continuing to attempt to identify a handful of commonly shared gene mutations in cancer. Clearly, the current concept of cancer is not consistent with the reality of the presence of high degrees of genetic diversity in patients. To solve this dilemma, cancer genome sequencing has been proposed to identify these common cancer genes, based on the assumption that cancer heterogeneity among patients is genetic “noise” and can be eliminated by validation using large patient samples. Unfortunately, this highly anticipated approach is delivering unwanted results.

In yet another example of heterogeneity, the vast majority of gene mutations are not shared among patients. In light of this disappointing fact, some have suggested shifting from gene identification to pathway characterization, others are searching for non-gene causes including bacterial/viral infections, cancer stem cells, metabolic stress and errors, oxidative stress, aneuploidy, inflammation, tumor/tissue interaction, immuno-deficiency, a large array of epigenetic effects. These different approaches represent essentially the same attempt to find common causative patterns but are now focused on different levels of genetic/epigenetic or cellular organization and their response under all kinds of environmental stress.

There is a need for a methodology for measuring system instability at the genome level.

There is additionally a need for measuring overall stress on the examined genome system.

There is also a need for identifying new types of chromosomal aberrations capable of monitoring condensation defects in cancer and genomic instability.

There is a further need for a system to characterize the main form of mitotic cell death, chromosomal fragmentation, caused by genome instability and drug treatments.

There is a still further need for re-evaluating the clinical usage of the ignored non-clonal chromosome aberrations, as the classification of all types of chromosome aberrations is important for genome based diagnosis and treatment options.

### SUMMARY OF THE INVENTION

The foregoing and other needs are fulfilled by this invention which provides, in accordance with a first diagnostic method aspect of the invention, a diagnostic method of determining tumorigenicity of a tissue specimen, or measuring the overall genome system instability of an individual patient. In accordance with the invention, there are provided the steps of:

determining a magnitude of genome diversity (i.e., the indicator of genome instability) in the tissue specimen; and

diagnosing a likelihood of cancer in response to said step of determining the magnitude of genome diversity.

In one embodiment of this method aspect, the step of determining the magnitude of genome diversity includes the step of determining the karyotypic heterogeneity in the tissue specimen.

In a further embodiment, the step of determining the presence of elevated genome diversity includes the step of detecting non-clonal chromosome aberrations (NCCAs). In some embodiments, the step of detecting NCCAs includes the further step of detecting the frequency of NCCAs. The step of diagnosing is responsive to said step of detecting the frequency of NCCAs.

In an advantageous embodiment of the invention, the step of detecting NCCAs includes the further step of screening lymphocytes.

Additionally, in a still further embodiment, the step of determining the presence of elevated genome diversity includes the step of applying Spectral Karyotyping or other cytogenetic methods to detect translocations and other structural and numerical alterations throughout the genome, including defective mitotic figures (DMFs) and chromosome fragmentation.

In accordance with a further method aspect of the invention, there is provided a diagnostic method of determining drug resistance of a patient. The further method includes the steps of:

determining the presence of genome diversity in the tissue specimen; and

diagnosing the drug resistance of the patient in response to said step of determining the presence of genome diversity.

In accordance with an additional method aspect of the invention, there is provided a screening method of determining a drug's effectiveness as well as determining the potential damage of genome instability. This further method application includes the steps of:

evaluating the effectiveness of inducing DMFs or chromosome fragmentation; and

evaluating the induced frequency of NCCAs.

A further application of the invention includes a method of measuring the overall system stress level. This additional method includes the step of using the frequencies of NCCAs to measure stress.

Cancer progression represents an evolutionary process where overall genome level changes reflect system instability and serve as a driving force for evolving new systems. To illustrate this principle it must be demonstrated that karyotypic heterogeneity (population diversity) directly contributes to tumorigenicity. Five well characterized in vitro tumor progression models representing various types of cancers were selected for such an analysis. The tumorigenicity of each model has been linked to different molecular pathways, and there is no common molecular mechanism shared among them. The common link of tumorigenicity between these diverse models is elevated genome diversity. Spectral karyotyping (SKY) was used to compare the degree of karyotypic heterogeneity displayed in various sublines of these five models. The cell population diversity was determined by scoring type and frequencies of clonal and non-clonal chromosome aberrations (CCAs and NCCAs). The tumorigenicity of these models has been separately analyzed. As expected, the highest level of NCCAs was detected coupled with the strongest tumorigenicity among all models analyzed. The karyotypic heterogeneity of both benign hyperplastic lesions and premalignant dysplastic tissues were further analyzed to support this conclusion. This common link between elevated NCCAs and increased tumorigenicity suggests an evolutionary causative relationship between system instability, population diversity, and cancer evolution. The present invention reconciles the difference between evolutionary and molecular mechanisms of cancer and suggests that NCCAs can serve as a biomarker to monitor the probability of cancer progression.

The approach of the present invention is to monitor system behavior, as there is no causative relationship between lower level parts within a complex system and it is impractical to monitor each change at various levels. It is therefore necessary, in accordance with the invention, to establish a system of using NCCAs to monitor the level of dynamics of the genome rather than specific changes at lower levels, as there are large numbers of specific changes at the lower molecular level that all can contribute to cancer formation or other types of complex diseases.

The chromosome represents not only the vehicle in which genes are carried but also serves as a genetic framework that controls all genes in a systemic manner. The karyotypic-defined genome is the driving force for cancer evolution and for other types of organismal evolution. Clinical cytogenetic analysis is commonly done in many types of cancers and often serves as a diagnostic and prognostic marker of cancer progression. Clonal events are recorded, but recent evidence shows that nonclonal events provide the diversity that is required for tumor progression and survival in the extremely harsh environment of a tumor. Change on the chromosomal level has the capability of directly altering the regulation of hundreds or possibly thousands of genes. Chromosomal change, including translocations, deletions, duplications, inversions, defective mitotic figures, and fragmentation of chromosomes can serve to increase diversity that is required for cancer progression and evolution.

The traditional approach of monitoring clonal chromosomal aberrations focuses on the same specific chromosomal changes shared by mitotic figures that are from the same individual or are the same chromosomal changes noted among different individuals. The traditional approach of using clonal aberrations was originally developed for detection in chromosome preparations from patients with leukemia. However, this type of approach is not applicable for solid tumors, because solid tumors display a high degree of heterogeneity and a high degree of genomic diversity (reflected as increased non-clonal aberrations). In addition, clonal aberrations do not display common patterns and are difficult to identify, usually developing only in the later stages of tumor formation.

The present inventive system focuses on monitoring NCCAs that are detected randomly, as a given NCCA occurs in only a single mitotic figure. Due to their apparent random nature and normally low frequencies. NCCAs have previously been considered as background artifacts. Now with the use of multiple color Spectral Karyotyping that can be used to monitor an entire genome by painting each individual chromosome coupled with the ability to check large numbers of mitotic figures, the inventors herein have recognized that the occurrence frequency of NCCAs is not random at all and has important implications. For example, data developed by the inventors has demonstrated that NCCAs could actually represent signature changes reflecting the level of increasing genomic instability that precedes and leads to cancer initiation and progression.

When the genome is unstable (due to defective DNA repair, checkpoint deficiencies, or cell stress that is induced by radiation or environmental chemical carcinogens), there appears to be a significantly increased rate of non-clonal chromosomal changes prior to any clonal aberration formation. This increased frequency of NCCAs reflects the unstable nature of the genome and may actually activate oncogenes or inactivate tumor suppression genes or change the gene expression profile for genes located on translocated chromosomes. Since there are many pathways that can lead to cancer development, and there are many different types of non-clonal chromosomal translocations, it is possible that many different combinations of NCCA events could initiate and/or promote cancer development, thus NCCAs are very likely an indicator of cancer transformation.

The new diagnostic system disclosed this application is based on two new features. First, there is disclosed herein the concept of using the frequency of NCCAs to monitor genomic instability. Second, there is disclosed the use of Spectral Karyotyping that is capable of detecting all translocations throughout the genome. Two types of new chromosomal aberrations have been characterized. These are DMFs and chromosome fragmentation, which have been overlooked for a several decades. Therefore, the establishment of this previously ignored approach of scoring NCCAs to monitor genomic instability is fundamentally important with practical applications particularly relevant for patients on chemotherapy.

The inventors herein identify four important practical usages of NCCAs. First, NCCAs have directly been linked to genomic instability by several important experiments. Most significant, it has been found that cancer cells display very high rates of NCCAs correlating with their level of genomic instability. This contention has been verified further through experiments using knock out mice that are engineered to be genomically unstable. These experiments verify that frequencies of NCCAs correlate with the level of overall genomic instability. Additional experimental support has been performed utilizing normal genomes by applying carcinogens that are known to cause instability, as well as using unstable cancer genomes, and also subjecting them to carcinogen treatments. Both of these experiments confirmed the direct correlation between the level of genomic instability and quantitatively measured rates of NCCAs. In all of these experiments, NCCAs represented the indicator of increasing genomic instability and provided the earliest detectable chromosomal changes preceding cancer initiation in solid tumors.

There is renewed interest in cancer genomic changes occurring at the chromosomal level. Study at the chromosomal level is a new trend for cancer research and is now considered the level at which genomic instability is reflected for the majority of cancers. Chromosomal biomarkers also have a distinct advantage primarily due to the fact that chromosomal changes represent an end product of multiple molecular pathways that generate high levels of heterogeneity typically seen in solid tumors. This will result in the ability to perform comparisons regardless of the molecular pathway or degree of heterogeneity. NCCAs are therefore useful to study genomic instability, which is a key feature of cancer development. In accordance with the present invention, genome instability should also contribute to other types of disease.

Second, NCCAs provide the underlying basis for cancer clonal evolution. This is significant as cancer research has been unable to provide an explanation or a theory for solid tumor progression. One of the most puzzling aspects is the lack of karyotype patterns displayed by similar or the same type of tumor and there is also no pattern of evolution during tumor progression. This has been a focus of intense investigation in the cancer research field, attempting to identify analogous patterns found in hematologically based cancers. NCCAs represent transient chromosomal alterations that ultimately lead to permanent changes that underlie cancer initiation. NCCAs mediate the chromosomal alterations that set off a particular pathway leading to the formation of clonal populations. Since this can occur through a variety of pathways, no common karyolype pattern has been found that leads to a pattern of clonal evolution in solid tumors. The apparent contradiction is that on the one hand, all NCCAs come from the same parental cell, while on the other hand NCCAs are different from the parental cells (some of them can be drastically different). When subsequent new clonal aberrations form, they appear not to be interconnected, however the inconsistent patterns seen in solid tumors can be explained by the occurrence of NCCAs. NCCAs therefore represent the important link between similar cancer phenotypes that display widely varying heterogeneity and are essential to the process of clonal evolution that activates cancer.

Third, the rate of NCCAs can be easily accessed from patients by screening lymphocytes derived from drawn blood samples rather than direct tissue biopsies. This makes the use of NCCAs a practical and feasible tool. The use of lymphocytes for the purpose of monitoring genetic susceptibility is not new. Lymphocytes have been used to detect chromosomal breakage as a means to correlate increased chromosomal instability in cancer patients. However, the rates of breakage are less reliable than rates of non-clonal translocations. Chromosomal breakage used to predict the likelihood of cancer was successful based on group data, but was not as reliable when used for individual based data. Levels of NCCAs can be used to monitor and establish a genomic instability baseline for each individual (since the genetic background is the same for all of an individual's cells). The use of a system to monitor genomic instability based on the use of NCCAs obtained from patient's lymphocytes is strongly supported by data.

Fourth, is the practical application of NCCAs as a biomarker capable of monitoring the potential of drug resistance and possible response of patients after chemotherapy. The correlation of high frequencies of NCCAs has been associated with the increased likelihood of drug resistance in cancer patients. The inventors have also surprisingly demonstrated that many of the current available chemotherapies can induce genome instability and thus also induce drug resistance. This new finding is very different from previously known mechanisms of bacterial antibiotic resistance. Therefore, the use of the frequencies and types of NCCAs is an important new method that can now be used to study drug resistance. NCCAs can thus be monitored in each patient to follow the affects of drug or reagent therapy and is a new method that can be used to assess underlying genomic instability and the potential for recurring cancer transformation. High rates of NCCAs are indicative of an unstable genome, the presence of which is a strong indicator of chemotherapy resistance.

An emerging genome-centric concept on cancer evolution states that overall genome level variation coupled with stochastic gene mutations serve as a driving force of cancer evolution by increasing the cell population diversity. The importance of non-clonal chromosome aberrations (NCCAs) (both structural and numerical) and their dynamic interplay with clonal chromosome aberrations (CCAs) in the immortalization process has been recently demonstrated and supports the genome-centric concept of cancer evolution. Similarly, the pattern of gene mutations within tumors occurs stochastically. These data and the absence of universal gene mutations revealed by recent large scale sequencing efforts reveals that genome dynamics and stochastic cancer evolution and its clinical implications should now be incorporated into the conceptual framework of cancer research.

Studies on clonal diversity and subsequent clinical outcomes in Barrett's esophagus reinforce the concept that cancer progression occurs through somatic evolution driven by genome instability coupled with an increase in or accumulation of clonal diversity. To date, however, most evolutionary analyses have focused on specific genetic loci rather than the overall genome level diversity. The impact of genetic variation at the genome level is much more profound than at the gene level, as the higher level of organization often constrains lower levels and displays more stable characteristics than lower levels. It is therefore expected that the major form of cellular population diversity is generated by karyotypic heterogeneity reflected as NCCA/CCA cycles (previously described as the waves of clonal expansion with the regeneration of genetic diversity in between) occurring during somatic evolution. It is thus more reliable and easier to measure the degree of diversity at the genome level than at the individual gene level. In addition, it has been a challenge to trace individual genes for most cancer types where there is a high level of genomic heterogeneity.

Increased NCCAs are associated with multiple genetic and environmental factors including dysfunction of genes that maintain genome integrity, over-expression of onco-proteins, exposure to carcinogens, cells reaching crisis stages prior to immortalization, etc. For a given cell population, elevated NCCAs will directly promote tumorigenicity. This correlation supports the biological significance of NCCAs in cancer formation. Previously, only the immortalization step was extensively shown to have such a correlation. To test the hypothesis further that increased levels of NCCAs directly promote tumorigenicity, it is necessary to link the two events in a simple model system.

There are a number of in vitro tumorigenicity models available. Most however, focus on the link between tumorigenicity and specific pathways rather than the evolutionary mechanism of tumorigenicity. Accordingly, a large number of pathways have been linked to tumorigenicity without revealing common mechanisms. In light of the discovery by the inventors herein that genome instability mediated somatic cell evolution is the common mechanism in cancer, some of the previously characterized systems have been reexamined with a focus on overall genome diversity rather than specific pathways. The inventors herein have selected five readily available in vitro models that represent various human and mouse cancer types to confirm that the linkage between increased levels of NCCAs and tumorigenicity represents a common feature across drastically different models transcending previously characterized molecular pathways.

In addition, the inventors have demonstrated that five patients with Gulf War Syndrome have displayed significantly higher levels of NCCAs, which illustrates the practical application of NCCAs and provides further support to the proof of principle.

FIG. 1 is a graphical representation of detected levels of NCCAs in individual patients plotted as a percentage of cells with chromosomal changes for patients that are normal patients or have been diagnosed with cancer or Gulf War Syndrome.

FIG. 2 is a graphical representation of the average and standard deviation of each group of patients as shown in FIG. 1.

In the field of cancer clinical cytogenetics only clonal chromosomal aberrations (CCAs) have been used to systematically characterize or “karyotype” cancers, this method is primarily based on experience with hematology cancers. Solid tumors behave differently than hematologically based cancers. There have been no universal clonal events identified in solid tumors; however NCCAS and unrelated CCAs have been reported. Previously, NCCAs have been disregarded as not significant, making it extremely difficult to reconcile cancer karyotype findings. In contrast, the inventors herein have demonstrated that not only are NCCAS significant but they also explain progressive heterogeneity and chemotherapy resistance. In addition, the quantitative frequencies of these structures can be used as a direct indicator of underlying latent genomic instability and the potential chemotherapy resistance and subsequent cancer transformation. Further, these structures represent fundamental underlying changes that precede cancer initiation and continue to occur during progression that likely represent the earliest detectable changes prior to tumor initiation.

The concept of NCCAs underlying cancer initiation and progression will change perspectives on certain aspects of cancer research. In particular, according to emerging trends, it is now known that chromosome aberrations can initiate cancer as opposed to being a consequence of cancer as was previously thought, and therefore, the focus will now shift to the examination of chromosomal alterations rather than the monitoring of specific pathways. This approach has more practical application since the present system can be used to monitor entire genomes, focusing on the earliest detectable changes. These structures can be applied for diagnostic and prognostic purposes as well as for monitoring drug effects on the genome.

Spectral karyotyping (SKY) was used herein to compare the degree of karyotypic heterogeneity displayed in various sublines of five in vitro systems, where the cell population diversity was determined by the frequency of NCCAs. The tumorigenicity of these models has been further analyzed to link elevated structural NCCAs and tumorigenicity. In addition, benign hyperplastic lesions (without evidence of carcinoma) were examined and displayed low levels of structural NCCAs. In contrast, premalignant dysplastic tissue of the c-myc transgenic mouse model displayed high levels of NCCAs. Based on the observations that there are many types of karyotypic aberrations, the distribution patterns of structural and numerical NCCAs as well as the contribution of various types of genome level variation to tumorigenicity have also been analyzed, suggesting the importance of using total frequencies of structural NCCAs when monitoring the potential tumorigenicity. Together, the present analysis agrees with the proposed model that chromosomal instability produces genetic variation and the more variation there is, the more likely a favorable combination will be produced that will result in a lesion that will produce malignancy/tumorigenicity. Thus, the identified common link between the elevated levels of NCCAs and increased tumorigenicity establishes a strong relationship between genome level diversity and tumorigenicity. Further, this information illustrates the relationship between the general evolutionary mechanism and large numbers of specific molecular mechanisms of cancer.

In brief, the evolutionary mechanism of cancer is equal to the collection of total number of individual molecular mechanisms. As each individual case often involves different molecular mechanisms and the mechanisms are constantly changing during cancer evolution, it is difficult to predict the status of cancer and the response to treatment based only on tracing specific pathways.

Another component of the present invention is the characterization of two new structures of NCCAs: defective mitotic figures (DMFS) and chromosome fragmentation. These two markers can also be used as clinical markers of progressive disease as well as to monitor the chemotherapy process.

It is clear that the new NCCA biomarker can be used; 1) to classify cancer patients in terms of the suitability of chemotherapy (i.e., to classify patients according to likely chemotherapy response and options, as some of the patients will be harmed if their genomes are not stable and they are subjected to chemotherapy); 2) to screen the general population to assess the risk of cancer, and early detection (the inventors have demonstrated that elevated NCCAs can be detected in the initial stages of cancer); 3) to further study the mechanism and patterns of cancer progression from the perspective of genomic instability; and 4) to screen the chemotherapeutical agents and environment insults.

The basic mechanism underlying these structures (NCCAs) is currently under study. There is an important direct correlation with the underlying genomic instability of cells (specifically tumor cells) that can be used to monitor and predict tumor behavior in clinical specimens. In addition to monitoring theses structures when examining tumor biopsy samples, these structures can also be easily obtained from blood lymphocytes in patients and, are therefore, useful practically to ascertain the inherent level of genomic instability of clinical patients that may be at risk for cancer or are in the initial stages of cancer to determine the likelihood of progression. NCCAs could be useful for clinical evaluation of treatment regimens by giving additional information to the clinician as to the possibility of progression, in addition to the stage of the tumor.

These structures also readily increase when known genomically destabilizing drugs or reagents are applied to cells of patient samples. Rates of NCCAs, therefore, could be used to determine the effects that a specific drug or chemotherapy agent has on tumor cells, i.e., will the drug in question increase or decrease genomic stability. Additionally, the application of certain chemotherapy agents to patient samples could determine the latent genomic instability level of a patient. The most genomically unstable patients will display the highest NCCA levels when a known destabilizing drug is applied. This process could become a very useful clinical tool for cancer that could also have applications in the toxicology field, as well as to screen chemotherapy agents to determine the extent of drug resistance.

### Experiments That Link the Increased NCCAs to Tumorigenicity

Various stages of cells representing the five models (Table 1) were briefly cultured. The original frozen cell passages used in the previous tumorigenicity studies were short-term cultured. After 2-4 days culture, mitotic cells were harvested for chromosome preparation. Briefly, cells were grown to 70% confluence and treated with colcemid for 4-8 h. Trypsinized cells were harvested and treated with hypotonic solution (0.4% KCL, 10 min at 37° C.), followed by Carnoy's fixation (3:1 of methanol and acetic acid) (three times at 20 min each) and air-dried. The chromosomal slides can be used for SKY immediately or stored at −70° C. for future use.

TABLE 1
Types and frequencies of various CCAs and NCCAs of the seven models analyzed

| | | | | |
|---|---|---|---|---|
| Cell Lines tissue | Chromosoma | | | |
| samples | 1 number | CCAs | sNCCAs (%) | Tumorigenicity |

The LNCap cell lines

| | | | | |
|---|---|---|---|---|
| Pd36: | 79.17 ± 14.35 | der(1)t(1; 15), | 30% | Low |
| | | der(6)t(4; 6), | | |
| | | der(4)t(4; 6; 10), | | |
| | | der(15)t(15; 1), | | |
| | | der(16)t(16; 6) | | |
| Pd69: | 90.39 ± 22.43 | der(1)t(1; 15), | 41.5% | |
| | | der(6)t(4; 6), | | |
| | | der(4)t(4; 6; 10), | | |
| | | der(15)t(15; 1), | | |
| | | der(16)t(16; 6), | | |
| | | der(13; 13) | | |
| Pd125: | 88.77 ± 19.97 | der(1)t(1; 15), | 53% | High |
| | | der(6)t(4; 6), | | |
| | | der(4)t(4; 6; 10), | | |
| | | der(15)t(15; 1), | | |
| | | der(16)t(16; 6) | | |

MCF10DCIS.com model

| | | | | |
|---|---|---|---|---|
| Pd9: | 48.35 ± 2.18 | der(1)t(1; 2), | 5.9% | Low |
| | | t(3; 17), t(17; 3), | | |
| | | der(6)t(6; 19), | | |
| | | der(9)t(9; 3; 5), | | |
| | | der(21)t(21; 17) | | |
| Pd29: | 49 ± 2.03 | der(1)t(1; 2), | 12% | |
| | | t(3; 17), t(17; 3), | | |
| | | der(6)t(6; 19), | | |
| | | der(9)t(9; 3; 5), | | |
| | | der(21)t(21; 17), | | |
| | | der(15)t(15; 21), | | |
| | | der(3)t(3; 9) | | |
| Pd46: | 47.58 ± 4.54 | der(1)t(1; 2), | 42% | High |
| | | der(6)t(6; 19), | | |
| | | der(9)t(9; 3; 5), | | |
| | | der(15)t(15; 21) | | |

MCF10-CSC model

| | | | | |
|---|---|---|---|---|
| CSC-MCF10A1: | 51.25 ± 14.29 | der(1)t(1; 13), | 24.3% | No |
| | | der(3)t(3; 9), | | |
| | | t(3; 17), t(17; 3), | | |
| | | der(6)t(6; 19), | | |
| | | der(9)t(9; 3; 5) | | |
| CSC-MCF10A2: | 51.82 ± 18.23 | der(1)t(1; 13), | 30% | No |
| | | der(3)t(3; 9), | | |
| | | t(3; 17), t(17; 3), | | |
| | | der(6)t(6; 19), | | |
| | | der(9)t(9; 3; 5) | | |
| CSC-MCF10A3: | 95.5 ± 22.00 | der(1)t(1; 13), | 42% | Yes |
| | | der(3)t(3; 9), | | |
| | | t(3; 17), t(17; 3), | | |
| | | der(6)t(6; 19), | | |
| | | der(9)t(9; 3; 5) | | |
| CSC-MCF10A4: | 48.96 ± 7.56 | der(1)t(1; 13), | 34.8% | No |
| | | der(3)t(3; 9), | | |
| | | t(3; 17), t(17; 3), | | |
| | | der(6)t(6; 19), | | |
| | | der(9)t(9; 3; 5) | | |

MCF10-HoxA1 model

| | | | | |
|---|---|---|---|---|
| HOXA1: | 46.55 ± 0.76 | der(3)t(3; 9), | 15.3% | Yes |
| | | der(9)t(5; 3; 9) + 1 | | |
| Control: | 46.5 ± 0.76 | der(3)t(3; 9), | 5.3% | No |
| | | der(9)t(5; 3; 9) + 1 | | |

Mouse ovarian cancer model

| | | | | |
|---|---|---|---|---|
| Pd9: | 71.08 ± 3.37 | der(10; 10) | 9.1% | No |
| Pd45: | 58.6 ± 13.21 | der(1)t(1; 2), | 30% | |
| | | der(8)t(8; 16) | | |
| Pd91: | 57.6 ± 13.89 | t(1; 2), t(8; 9), | 50% | Yes |
| | | t(5; 3), t(3; 2) | | |

MCF10-Rad6B (benign lesion)

| | | | | |
|---|---|---|---|---|
| MC15 | 52.17 ± 16.18 | der(1)t(1; 2), | 4.3% | No |
| | | der(1)t(1; 5), | | |
| | | t(3; 17), t(17; 3), | | |
| | | der(6)t(6; 19), | | |
| | | der(9)t(5; 3; 9) | | |

Myc-transgenic mouse model (premalignant dysplastic tissue)

| | | | | |
|---|---|---|---|---|
| MG2 | 40.41 ± 5.16 | −17 | 24% | Yes |

For each sample of these models, an average of 50 SKY images were analyzed. For the MCF10-HoxA1 model, in addition to the listed frequency of structural NCCAs, 78% of errors in segregation reflected by the sticking chromosomes were detected in the HoxA1 line, while 14% of errors were detected in the control cell line.

MCF10A-Rad6B clone 5 cells were derived by stable transfection of Rad6B, a fundamental component of postreplication DNA repair pathway. MCF10-Rad6B clone 5 cells (1×107) were suspended in Matrigel and injected into the mammary fat pads of female immunodeficient nude mice, and lesions from the injection sites were harvested at 70 days. Harvested xenografts were cultured in DMEM/F12 supplemented with 5% horse serum, 10 μg/ml insulin, hydrocortisone and 10 ng/ml EGF to derive MC15. MC15 cells were harvested and chromosomes prepared within 2-4 passages for SKY analysis.

Chromosome preparation from proliferating mammary glands of MMTV-c-myc transgenic mice Proliferating mammary glands were collected from two virgin female MMTV-c-myc transgenic mice at age of 7 months. Virgin females of this transgenic line of mice spontaneously develop palpable mammary tumors at ages of 7-9 months. The proliferating mammary glands used in this study were collected from an area distant from a palpable tumor, and histology of the glands in the same area showed only proliferating glands without atypia. Proliferating glands were briefly cultured and chromosomes were prepared for SKY analysis.

Sky and Data Analysis

Following probe denaturation, hybridization and SKY detection, randomly selected mitotic figures were photographed and analyzed by SKY imaging software. Fifty to hundred SKY images were captured for each cell population to identify commonly shared karyotype features and to reveal the karyotypic diversity of these various cell populations. NCCAs were scored by identifying chromosomal numbers, chromosome translocations/large deletions or other types of abnormality detected within a given mitotic cell. There are two steps needed to score frequencies of NCCAs and CCAs. First, a 4% cutoff line is used to identify any specific recurrent karyotypes or CCAs. The frequency of a CCA is determined by calculating the number of cells displaying the same CCA divided by the total cells examined (50-100). Non-clonal karyotypes (NCCAs) are classified as having a frequency lower than 4%. The total frequencies of NCCAs of a given cell population is then calculated by using all cells displaying NCCAs divided by the total cells examined. Both types of CCAs as well as frequencies and types of NCCAs are listed in Tables 1 and 2.

TABLE 2
Distribution of various types of structural NCCAs for MCF10-CSC model

| | | |
|---|---|---|
| | | Frequencies |
| Cell lines | Recorded abnormal structures | (%) |

MCF10A-CSC-1 (# of karyotypes = 53)

| | | |
|---|---|---|
| t-NCCA | 1 | 1.9% |
| DMF | 7 | 13.0% |
| Other abnormal images | 5 | 9.4% |
| | Total | 24.3% |

MCF10A-CSC-2 (# of karyotypes = 60)

| | | |
|---|---|---|
| New CCA | der(15; 22) | 6.7% |
| t-NCCAs | 3 | 5.0% |
| Chr-F | 3 | 5.0% |
| DMF | 6 | 10.0% |
| Other abnormal images | 6 | 10.0% |
| | Total | 30.0% |

MCF10A-CSC-3 (# of karyotypes = 50)

| | | |
|---|---|---|
| New CCA | der(15; 22) | 8.0% |
| t-NCCAs | 5 | 10.0% |
| Chr-F | 3 | 6.0% |
| DMF | 10 | 20.0% |
| Other abnormal images | 3 | 6.0% |
| | Total | 42.0% |

MCF10A-CSC-4 (# of karyotypes = 60)

| | | |
|---|---|---|
| New CCA | der(15; 22) | 10.0% |
| | der(13; 22) | 5.0% |
| t-NCCAs | 5 | 8.3% |
| Chr-F | 7 | 11.6% |
| DMF | 7 | 11.6% |
| Other abnormal images | 2 | 3.3% |
| | Total | 34.8% |

t-NCCA refers to translocated chromosomes.
Chr-F refers to chromosome fragmentation.
Other abnormal images refer to these previously uncharacterized mitotic aberrations.

### In Vivo Tumorigenicity Test

In earlier studies, it was learned that all cigarette smoke condensate (CSC)-treated MCF10A cells efficiently formed colonies in soft-agar. The inventors then re-established cell lines from the soft-agar colonies and further examined the persistence of their transforming characteristics. The re-established cell lines, when plated after 17 passages without CSC treatment, still formed colonies in soft-agar. To determine whether the cell lines showing transformed characteristics in the anchorage-independent assay can grow in nude mice, the inventors injected four selected CSC-transformed cell lines, MCF10A-CSC1, MCF10A-CSC2, MCF10A-CSC3, and MCF10A-CSC4 into female nude (nu/nu) mice (with 105 cells of each cell line suspended in Matrigel) (BD Biosciences, San Diego, Calif.). Palpable tumors appeared in 20 days and animals were sacrificed in 44 days.

### Statistical Analysis

Ninety-five percent confidence intervals were calculated by combining the lines with the highest and the lowest tumorigenicity for each model. A Student's t-test was then run on this data showing a significant difference in NCCA levels between cells with high and low tumorigenicity (P=0.01791) (see, FIG. 11A). Ninety-five percent confidence intervals of chromosome number were also calculated for each cell line studied (see, FIGS. 11B-11F).

The reproducibility of NCCA level is very high. Though many factors can influence NCCA frequency including culture conditions and genetic makeup of a given cell line, the frequency of NCCAs is reproducible for a similar group. For example, in the MCF10 breast disease model, duplicates of treated and untreated show a significant difference (P=0.00055) in NCCA frequency when the treated are compared to the untreated, however standard deviation within treatments is quite low (0.00212132 in treated and 0 in untreated). Similarly, when comparing two stages of the immortalization process of the Li-Fraumeni model, duplicates of the earliest stage were similar (SD=0.008485281) and significantly different from the duplicates of later stages of the cell populations (P=0.0034). Similar results were reported regarding the frequencies of NCCAs in ATM−/− mice as well as various cancer cell lines with or without onco-protein expression.

### Results Linking NCCAs to Tumorigenicity

Molecular characterizations and measured genome diversity for the five models The molecular characterization of these five models has been accomplished by previous studies and the key points are briefly summarized (Table 3, below). To examine genome diversity, multiple color SKY was used to score the level of NCCAs and types of CCAs. The following is detailed information on each model.

### The LNCaP Model

A unique prostate cancer model with three distinctive stages has been developed using sublines of LNCaP cells originally established from a human prostate adenocarcinoma. Within this model, C33 (passage number<33) represents the early stage that is androgen-responsive; C51 (passage 45-70) represents the middle stage with decreased androgen-responsiveness; and C81 (passage 81-120) represents the late stage with androgen-unresponsiveness and increased tumorigenicity, illustrated by a xenograft animal model, where C33 and C81 stage cells of the LNCaP cell model showed differential tumorigenicity when implanted subcutaneously in nude mice. In this model increased genetic aberrations, such as microsatellite instability and allelic loss were observed in later passages, but the karyotypes appeared to be stable throughout the progressive transformation. This illustrates the link between tumorigenicity and increased genetic alterations reflected by microsatellite instability and chromosomal allelic loss.

Three cell populations representing C33 (pd36), C51 (pd69), and C81 (pd125) were used for SKY analysis. The overall karyotypes of all cells at various passages shared the same set of five altered chromosomes demonstrating the overall stability at the karyotypic level as determined by the presence of stable CCAs (FIG. 8). At pd69, der(13;13) formed as a new transitional CCA, however, it was lost by pd125 (Table 1). Thus there were no specific late passage CCAs. Increased structural NCCAs, on the other hand, represent a significant feature of the transition between early and later passages.

FIGS. 8A and 8B illustrate examples of increased levels of NCCAs detected from the late stages of in vitro models coupled with increased tumorigenicity, specifically a karyotype comparison between an early stage (p36) (FIG. 8A) and a late stage (p105) (FIG. 8B) of the LNCaP cell line. In addition to sharing all four types of CCAs as indicated by the blue colored boxes, there are more NCCAs detected as indicated by the yellow boxes coupled with increased tumorigenicity.

Increasing level of NCCAs combined with progressing cell passages clearly correlates with increased tumorigenicity. The fact that C33, which exhibits delayed tumor formation also has a relatively high degree of NCCAs (30%), further supporting the notion that increased levels of NCCAs promote tumorigenicity. From an evolutionary viewpoint, the higher the frequency of NCCAs increases the probability of cancer progression in shorter periods of time. Tumorigenicity, can be achieved with lower frequencies of NCCAs but requires longer time frames for the selection process to occur.

### The MCF10DCIS.com Model

MCF10DCIS.com xenograft is a model of human comedo ductal carcinoma in situ. This cell line was cloned from a cell culture initiated from a xenograft lesion obtained after two successive trocar passages of a lesion formed by premalignant MCF10AT cells. Early passage cells display a less invasive capability while the late-passage cells have a more extensive invasive capability. Various passages of this cell line were SKY analyzed to identify karyotype patterns as shown in Table 1.

The majority of the altered chromosomes were shared among the three passages examined.

With passage progression, dynamic NCCAs and CCAs were evident with some CCAs being replaced by others. At passage pd46, in addition to increased NCCAs, even the retained CCAs were not evenly distributed throughout the population indicating a high degree of heterogeneity as the degree of homogeneity drops. At passage pd46, der(15)t(15;21) were newly formed and high levels of NCCAs observed, thus linking these changes to increasingly invasive phenotypes. The mechanism of highly aggressive phenotypes was recently linked to stromal-epithelial interaction.

### The MCF10 Model Transformed by Cigarette Smoke Condensate (CSC)

To exclude the possibility that a specific CCA such as der(15)t(15;21) play a major role in the increased tumorigenicity observed in the MCF10DCIS model, it would be ideal to use cell populations that display different degrees of tumorigenicity and yet share the same marker chromosomes (identical CCAs). Four transformed lines have been generated by treatment with CSC, independent of the MCF10DCIS.com model. Even though all four lines displayed anchorage-independent growth in soft-agar, there was only one line that generated tumors in immunodeficient mice (see tumorigenicity session). Comparison of the karyotypic features of these four transformed lines showed they share six altered chromosomes in common (FIG. 9). Three of the alterations are shared in common with MCF10DCIS indicating the same origin for these two differently transformed systems (see, Table 1).

FIGS. 9A and 9B illustrate example of increased levels of NCCAs detected from the late stages of in vitro models coupled with increased tumorigenicity, including the comparison between subline MCF10A-CSC-1 (FIG. 9A) and CSC-3 (FIG. 9B). Both lines share five common types of CCAs as indicated by boxes 910. In line CSC-3 with increased tumorigenicity, in addition to ploidy changes, there were many NCCAs detected as indicated by boxes 920.

Although the four lines displayed the same sets of altered CCAs, NCCAs occurred at different levels in these lines. Various types of structural and numerical NCCAs are listed in Table 2. As illustrated by the tumorigenic assay of immunodeficient mouse xenografs, only CSC-MCF10A3 produced tumors in immunodeficient mice. In addition to elevated levels of NCCAs, the average chromosome number was also increased in CSC-MCF10A3. Therefore, in this system, increased ploidy and the frequency of NCCAs were linked to tumorigenicity.

### The MCF10 Model Transformed by HOXA1

To exclude the possibility that ploidy rather than a high degree of diversity contribute to the tumorigenicity that is observed with CSC-MCF10A3, an additional subline was selected with identical karyotypes (and ploidy status) but these lines displayed a diversity of NCCAs. This subline was obtained by spontaneously transforming MCF10 cells by over-expression of HOXA 1. Human growth hormone-regulated HOXA 1 has been shown to be a mammary epithelial oncogene. HOXA 1 stimulates the transcriptional activation of a number of pro-oncogenic molecules including cyclin D1 and Bcl-2 that promotes proliferation and survival. Over-expressed HOXA 1 in human mammary carcinoma cells results in drastically increased tumorigenicity.

FIGS. 10A, 10B, and 10C illustrate examples of increased levels of NCCAs detected from the late stages of in vitro models coupled with increased tumorigenicity. This figure shows the comparison between the HOXA1 expressed line and the control line generated from MCF10. Both lines displayed the same karyotypes with two identical CCAs indicated by the boxes 1010 in FIG. 10A. Interestingly, however, the HOXA1 line also displays a much higher level of abnormal mitotic figures (chromosomes are not well condensed), by a arrow 1020 or separated (indicated by arrows 1025 in FIG. 10B. These defective mitotic figures are types of NCCAs.

The degree of genome diversity of the cell line over-expressing HOXA1 (stable transfected with HoxA1 expression plasmid) and the control cell line containing vector only (Table 1) were compared. Both the HOXA1 line and the control line shared identical marker chromosomes and the karyotypes were identical (see, FIG. 10). The major difference was the frequency of defective mitotic figures (DMFs), a new phenotype of chromosome condensation defects and G2-M checkpoint deficiencies. In addition, the frequency of errors in cell division that are related to DMFs was higher in the HOXA1 line (FIG. 10). DMFs represent an ignored karyotypic aberration. The key description of a DMF is its differential condensation among all chromosomes and its genetic consequences causing an increase in population diversity and possibly leading to typical chromosomal aberrations such as aneuploidy, deletion, or translocations. As DMFs are a typical form of NCCA, the high frequencies of DMFs observed from the HOXA1 line indicates a high degree of genome diversity. Thus both the involvement of the HOXA1 oncogene and elevated NCCAs were co-linked to tumorigenicity.

FIGS. 11A-11F illustrate the distribution of structural and numerical NCCAs. In FIG. 11A, the distribution of NCCAs across the five cell lines of five in vitro models with the highest tumorigenicity and the five cell lines with the lowest. The bars indicate 95% confidence intervals. The difference between high and low tumorigenicity is significant (P=0.01791, Student's t-test), illustrating the significant relationship between frequencies of NCCAs and tumorigenicity. In FIGS. 11B to 11F, the distribution of chromosome number across the five systems analyzed. The graphs represent average chromosome number, bars indicate 95% confidence intervals. A change in the chromosome number does not associate with increased tumorigenicity in most lines except MCF10-CSC, possibly due to the ploidy. Passages/cell lines with higher tumorigenicity, however, tend to show increased confidence interval widths indicating more variance in chromosome number.

### Mouse Ovarian Cancer Model

Mouse syngeneic ovarian cancer models have been established and have proven to be very useful in the study of temporal molecular and cellular events during neoplastic progression. Primary mouse ovarian surface epithelial cells were isolated and cultured for varying generations. It is known that tumorigenicity (tested in nude mice) rises with increasing passage number. Three representative stages of a parallel experiment were selected for karyotype analysis representing pd9, pd45, and pd91 (see, Table 1).

Even at an early stage (passage 9), the karyotypes were clearly no longer normal as the population of cells contained 10% NCCAs and a CCA [der(10; 10)]. This initial CCA was replaced by two new CCAs der(1)t(1; 2), der(8)t(8; 16). Only der(1)t(1; 2) was detected during the later stages, illustrating karyotypic dynamics during in vitro culture. Again, the most prominent feature linking the cell progression stages was the percentage of NCCAs. During early passages NCCAs were detected in only 10% of all cells analyzed. By passage 91, however, NCCAs were detected in almost all cells, even though these cells also contained a four CCAs. Thus, the elevated NCCAs and two clonal aberrations were linked to tumorigenicity. In a parallel experiment, the tumorigenicity of an independent cell culture series was linked to increased numerical NCCAs (aneuploidy) and no recurrent CCAs were detected and distinct remodeling of the actin cytoskeleton and focal adhesion complexes were coupled with down-regulation and/or aberrant subcellular location of E-cadherin and connexin-43.

### Tumorigenicity Analysis

In order to establish a strong relationship between the level of NCCAs and tumorigenicity, cells with different levels of NCCAs were injected into mice and then comparatively analyzed for tumorigenicity. In most of these models, the tumorigenicity of various stages of the cell populations was previously tested using this assay and the data are readily available. To reduce variation in the present analysis, the original frozen cell passages used in the tumorigenicity studies were used in the present SKY analysis. Since the relative levels of NCCAs detected should be similar among these cells including those used to test tumorigenicity, the detected occurrence of increased NCCA frequencies should take place prior to injection into animals. As illustrated in FIG. 11 and Table 1, in each model, the highest tumorigenicity was always associated with the highest frequencies of structural NCCAs. Interestingly, in the LNCaP prostate cancer model, compared to early passage cells, the late stage cells with androgen-unresponsiveness, produced tumors two times faster, while the frequencies of NCCAs nearly doubled between early and late stage cells.

The tumorigenicity of the MCF10A-CSC model was then examined. The control MCF10A cells as well as three of the CSC-transformed cells lines (MCF10A-CSC1, CSC-2, and CSC-4) did not form tumors in the nude mice within 20 days, even though all CSC lines exhibit anchorage-independent growth. Only the MCF10A-CSC3 cell line grew and formed palpable tumors in the nude mice within 20 days. Thus, tumorigenicity is linked to the highest level of genome diversity. In conclusion, for all five models, the highest levels of genome diversity were linked to tumorigenicity.

### BRIEF DESCRIPTION OF THE DRAWING

Comprehension of the invention is facilitated by reading the following detailed description, in conjunction with the annexed drawing, in which:

FIG. 1 is a graphical representation of detected levels of NCCAs in individual patients plotted as a percentage of cells with chromosomal changes for patients that are normal patients or have been diagnosed with cancer or Gulf War Syndrome;

FIG. 2 is a graphical representation of the average and standard deviation of each group of patients as shown in FIG. 1;

FIGS. 3A, 3B, 3C, and 3D are photographic representations that are useful to illustrate morphologic features of chromosome fragmentation;

FIGS. 4A, 4B, 4C, and 4D are photographic representations that are useful to illustrate chromosome fragmentation that results in cell death;

FIGS. 5A, 5B, 5C, and 5D are photographic representations that illustrate TUNEL staining performed on cells undergoing chromosome fragmentation to show negative staining of cells displaying chromosome fragmentation and to illustrate that chromosome fragmentation is not apoptotic;

FIG. 6 is a graphical representation that illustrates that the chromosome fragmentation rate is associated with genomic instability;

FIG. 7 is a representation of a model that illustrates the relationship between chromosome fragmentation and other types of cell death;

FIGS. 8A and 8B illustrate examples of increased levels of NCCAs detected from the late stages of in vitro models coupled with increased tumorigenicity, specifically a karyotype comparison between an early stage (p36) (FIG. 8A) and a late stage (p105) (FIG. 8B) of the LNCaP cell line;

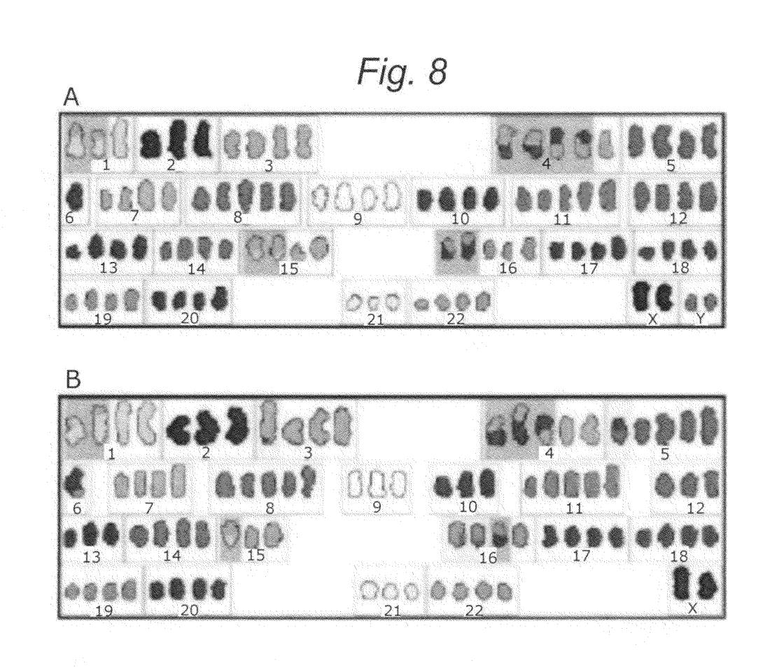

FIGS. 9A and 9B illustrate example of increased levels of NCCAs detected from the late stages of in vitro models coupled with increased tumorigenicity, including the comparison between subline MCF10A-CSC-1 (FIG. 9A) and CSC-3 (FIG. 9B). Both lines share five common types of CCAs as indicated by the blue colored boxes;

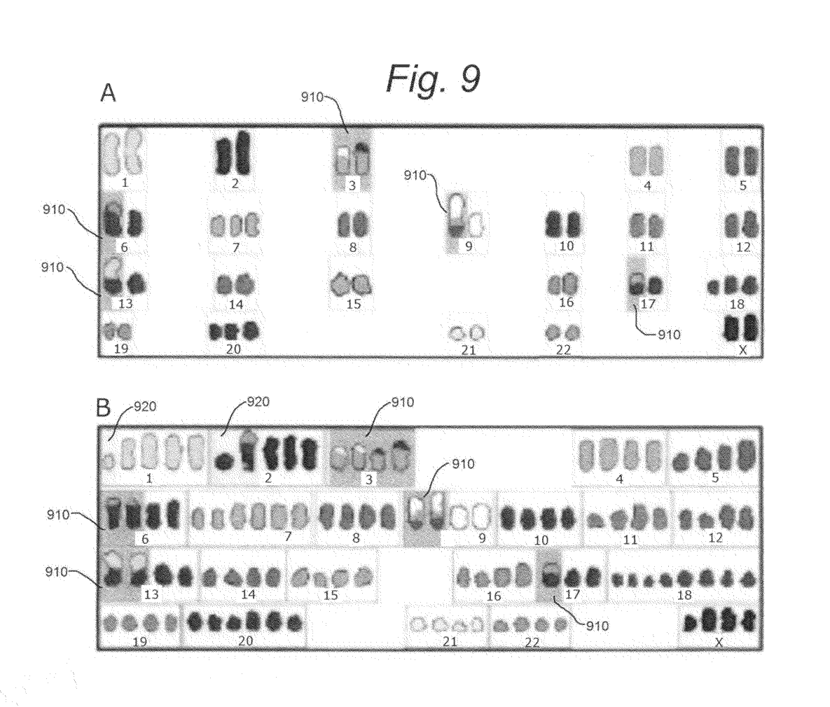

FIGS. 10A, 10B, and 10C illustrate examples of increased levels of NCCAs detected from the late stages of in vitro models coupled with increased tumorigenicity, specifically the comparison between the HOXA1 expressed line and the control line generated from MCF10, both of which displayed the same karyotypes with two identical CCAs;

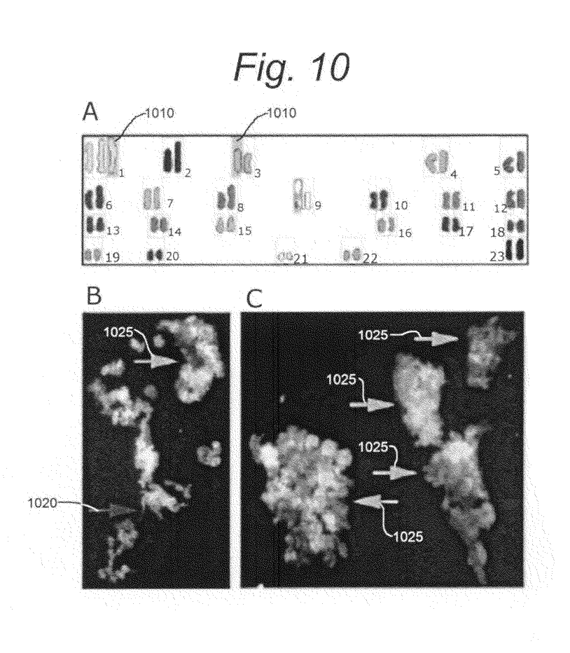

FIGS. 11A-11F illustrate the distribution of structural and numerical NCCAs linking to tumorigenicity;

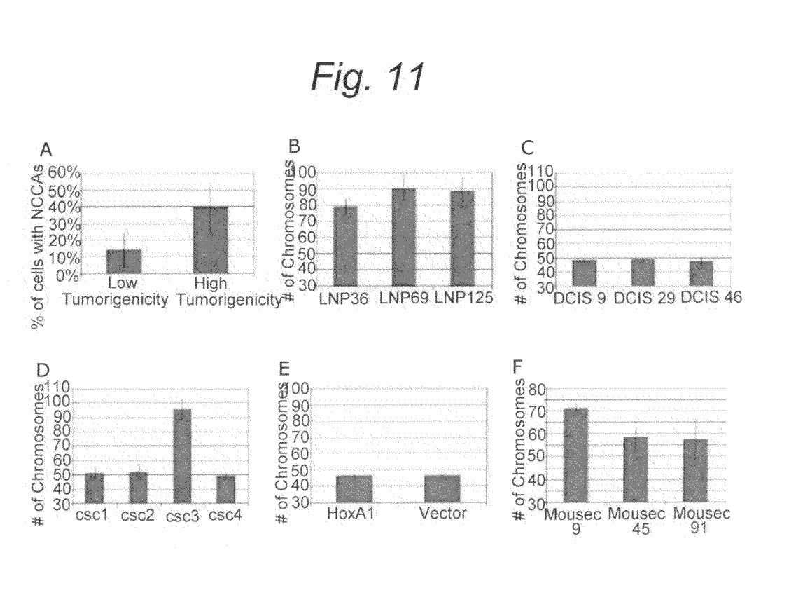

FIG. 12 illustrates the evolutionary mechanism of cancer and its relationship with molecular mechanisms; and

FIG. 13 illustrates the interactive relationship between multiple levels of heterogeneity and types of evolutionary selection.

### DETAILED DESCRIPTION

The following is a detailed description of a new type of NCCA and of a new type of mitotic cell death, termed “chromosome fragmentation,” which is a consequence of certain cellular stressors such as inherited genomic instability or chemotherapeutic treatment in M phase, and a pathologically related process that results in the breakdown of the chromosomes, elimination of genetic material, and subsequent death of cells. This form of cell death is different from typical apoptosis and mitotic catastrophe. It is caspase independent, does not exhibit the typical oligosomal DNA degradation of apoptosis, and is not inhibited by overexpression of Bcl-2.

Classic methods of inducing mitotic catastrophe do not increase levels of chromosome fragmentation detectable by cytogenetic analysis. Chromosome fragmentation, although morphologically similar to, is distinct from, S-phase premature chromosome condensation (and chromosome pulverization) as chromosomes undergoing fragmentation are from mitotic, nonreplicating cells. Chromosome fragmentation offers insight into the basis for chromosome pulverization in cases where the genome has been destabilized. Chromosome fragmentation represents a clinically relevant form of cell death, which unlike other types of cell death is clinically identifiable using standard cytogenetic analysis that is commonly performed on tumors, due to its defined morphologic features. Chromosome fragmentation is an important form of non-clonal chromosome aberrations (NCCAs), which are linked to cancer progression. It can serve as a much needed biomarker of induced cell death and genome instability.

### Materials and Methods of Studying Chromosome Fragmentation

Cell death plays a key role for both cancer progression and treatment. In this disclosure the inventors characterize chromosome fragmentation, a new type of cell death that takes place during metaphase where condensed chromosomes are progressively degraded. It occurs spontaneously without any treatment in instances such as inherited status of genomic instability, or it can be induced by treatment with chemotherapeutics. It is observed within cell lines, tumors, and lymphocytes of cancer patients. The process of chromosome fragmentation results in loss of viability, but is apparently nonapoptotic and further differs from cellular death defined by mitotic catastrophe. Chromosome fragmentation represents an efficient means of induced cell death and is a clinically relevant biomarker of mitotic cell death. Chromosome fragmentation serves as a method to eliminate genomically unstable cells. Paradoxically, this process could result in genome aberrations common in cancer. The characterization of chromosome fragmentation may also shine light on the mechanism of chromosomal pulverization.

The present invention constitutes a method is, as noted, useful for the detection of cancer (both status of individual system instability and its potential response to treatment) as well as understanding the mechanism of cancer progression. There is herein described a new type of mitotic cell death, termed “chromosome fragmentation,” which is a consequence of certain cellular stressors such as inherited genomic instability or chemotherapeutic treatment in M phase, and a pathologically related process that results in the breakdown of the chromosomes, elimination of genetic material, and subsequent death of cells. The somatic evolution of cancer is similar to natural evolution with system stability mediated genetic heterogeneity playing a key role.

Cell culture. Cell lines used by the inventors herein include HeLa, HCT116, HCT116 p53−/−, HCT116 14-3-3s−/−, H460-neo, H460-Bcl-2, and H1299-v138. HCT116 14-3-3s−/− cells were grown in McCoy's 5A medium; others were grown in RPMI 1640. G418 (400 μg/mL) was used as necessary.

Induction of chromosome fragmentation. After 3 to 8 h treatment of colcemid, mitotic cells were gently shaken off and resuspended in culture medium containing colcemid. Cells were then treated with doxorubicin or methotrexate at 1 μg/mL for various times.

Cytogenetic examination. Cells were harvested and cytogenetic slides were prepared using standard protocols. Slides were stained by Giemsa for scoring nuclear structures or stored at −20° C. for further characterization including spectral karyotyping (SKY) and antibody staining. The fragmentation index was determined by dividing the number of cells displaying chromosome fragmentation by the total number of mitotic cells, including cells undergoing chromosome fragmentation.

Spectral karyotyping analysis. SKY was performed on mitotic spreads as herein described. Briefly, cytogenetic slides were denatured and hybridized with human painting probes. After washing and spectral karyotyping detection, mitotic structures were captured using a charge coupled device camera.

Bromodeoxyuridine incorporation and antibody staining. Cells were pretreated for 3 h with colcemid and collected via mitotic shake off. Cells were treated for 6 h with 1 μg/mL doxorubicin, colcemid, 100 μmol/L deoxycytidine, 100 μmol/L bromodeoxyuridine (BrdUrd), and harvested for cytogenetic analysis.

Antibody staining was performed as herein described. Cell suspensions were dropped on ice-cold microscope slides followed by washes in cold buffer [10 mmol/L Na2HPO4, 0.15 mol/L NaCl, 1 mmol/L EGTA, and 0.01% NaN3] and buffer [1.0 mmol/L triethanolcomine-HCl (pH 8.5), 0.2 mmol/L NaEDTA (pH 8.0), 25 mmol/L NaCl, 0.05% TweenR20, and 0.1% bovine serum albumin]. Slides were then air dried and primary antibodies were loaded.

Viability assessment. Viability was assessed using a LIVE/DEAD assay (Invitrogen) according to the manufacturer's protocol.

Terminal deoxyribonucleotide transferase-mediated nick-end labeling (TUNEL) staining was performed using a kit supplied by Roche. Briefly, mitotic cells were induced to undergo chromosome fragmentation washed thrice in PBS, attached to slides using a Shandon cytospin at 500 rpm for 5 min, fixed in 4% paraformaldehyde, and subjected to TUNEL.

Caspase inhibition. Caspase activity was inhibited by treatment of HCT116 cells for 12 h with 20 mmol/L z-vad-fmk followed induction of chromosome fragmentation (by concurrent treatment with colcemid and 1 μg/mL doxorubicin) for 6 or 12 h in the presence of or after removal of z-vad-fmk. Following treatment, cytogenetic slides were made and scored.

Caspase-3 activity. Caspase-3 activity was measured using the caspase-3 colorimetric assay kit from R&D Systems as herein described. HCT116 cells were treated for 8 h with colcemid with and without z-vad-fmk. Mitotic cells were then shaken off and treated with doxorubicin for an additional 6 h and 12 h.

Western blotting. Analysis was performed as herein described.

Induction of genomic instability. For induction of genomic instability, H1299-v138 cells were grown at 39° C. for 2 weeks and then grown at 32° C. where cells were photographed and collected daily and cytogenetic preparations were made and analyzed for chromosome fragmentation.

Induction of mitotic catastrophe. Mitotic catastrophe was induced as herein described. HCT116 14-3-3−/− cells were treated for up to 72 h with 2 μg/mL doxorubicin and HCT116 p53−/−. The cells were treated with 1 μg/mL aphidicolin for up to 96 h and were each harvested every 24 h. Cytogenetic preparations and cytospin preparations were made allowing for chromosomal and morphologic analysis.

### Results of Chromosome Fragmentation Studies

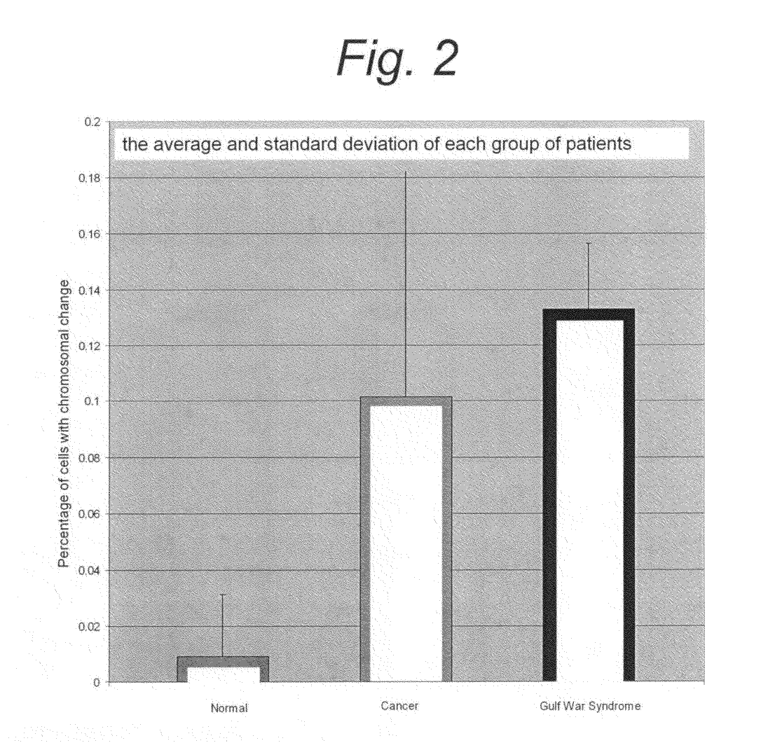

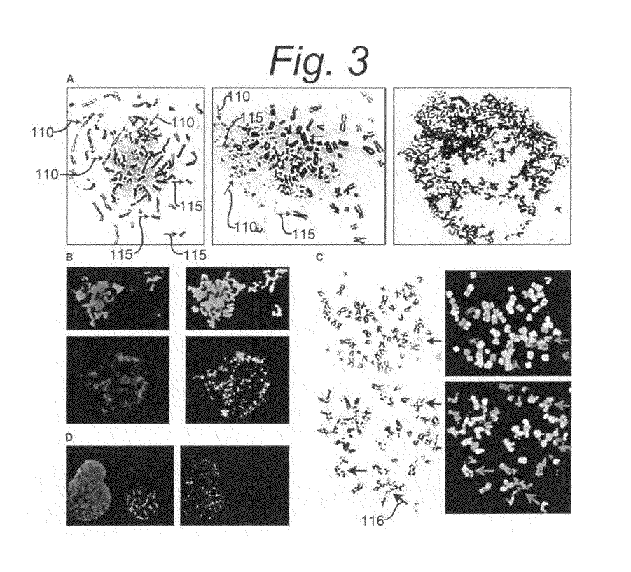

Chromosome fragmentation represents a unique phenotype of chromosome aberration. To define chromosome fragmentation, the morphology first is characterized. Fragmented chromosomes are distinct from normal chromosomes prepared by cytogenetic techniques. Chromosomes that are progressively cut into smaller pieces and fragmented chromosomes often show lighter density of Giemsa or 4′,6-diamidino-2-phenylindole (DAPI) staining than normal chromosomes stained in parallel indicating the loss of chromosomal material. Mitotic figures displaying chromosome fragmentation can be grouped into at least three groups, i.e., early fragmentation where few chromosomes are broken, midstage fragmentation where a significant number of the chromosomes have been fragmented, and late stage where all or most of the chromosomes have been fragmented, suggesting a progressive process (see, FIG. 3A). Chromosome fragmentation is not the result of the slide preparation, as under identical conditions both normal and fragmented chromosomes coexist within the same cell (FIG. 3A). In fact, the variable frequencies of fragmented chromosomes are determined by the specific cell line used and drug treatment before the making of slides (FIG. 2D).

FIGS. 3A, 3B, 3C, and 3D are photographic representations that are useful to illustrate morphologic features of chromosome fragmentation. Chromosomes undergoing fragmentation display many breaks and often seem frayed. In FIG. 3A, Giemsa staining shows chromosome fragmentation is a progressive process with early stages showing few fragmented chromosomes [left, chromosome fragmentation (arrows 110); intact chromosomes (arrows 115)], mid stage with approximately half of the chromosomes fragmented (middle), and late stage with nearly all chromosomes except for one at the top showing degradation (right). In FIG. 3B, there is illustrated evidence that chromosome fragmentation occurs in condensed mitotic chromosomes. Cells undergoing chromosome fragmentation stain positive (left DAPI, right FITC) for phosphorylated H3 (Ser10) both in early (top) and late (bottom) stages.

In FIG. 3C, spectral karyotyping images (inverted DAPI right, SKY left) indicate that chromosomes are condensed before fragmentation as fragmented chromosomes retain their chromosomal domains in early and later stages of fragmentation (top and bottom). Additionally, spectral karyotyping images show that single chromosomes can be eliminated via chromosome fragmentation (arrow 116).

In FIG. 3D, chromosome fragmentation is distinct from S-phase premature chromosome condensation as chromosomes from S-phase cells forced to undergo premature chromosome condensation should show BrdUrd uptake (DAPI left, anti-BrdUrd-FITC right). Cells undergoing chromosome fragmentation (cell on right) do not show incorporation of BrdUrd, whereas S-phase cells (left cell) from the same treatment do.

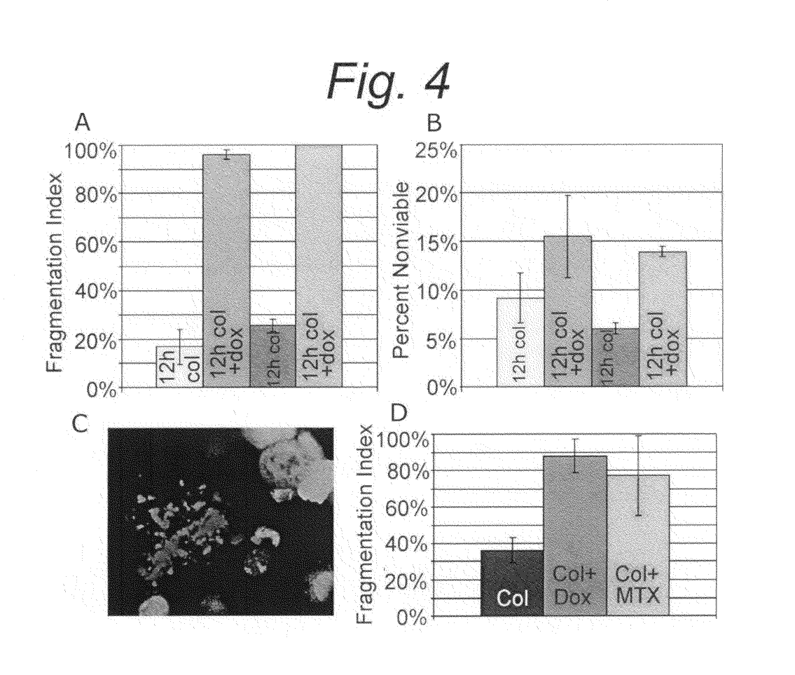

FIGS. 4A, 4B, 4C, and 4D are photographic representations that are useful to illustrate chromosome fragmentation that results in cell death. As the rate of chromosome fragmentation increases (FIG. 4A), cellular viability decreases (FIG. 4B). Viability staining shows that cells undergoing chromosome fragmentation display loss of membrane integrity as ethidium homodimer is able to enter the cell and stain chromosome fragments red (FIG. 4C). Viable cells show only green staining. Chromosome fragmentation is induced more efficiently if combinations of chemotherapeutics and microtubule inhibitors are used (FIG. 4D). Col, colcemid; dox, doxorubicin; MTX, methotrexate.

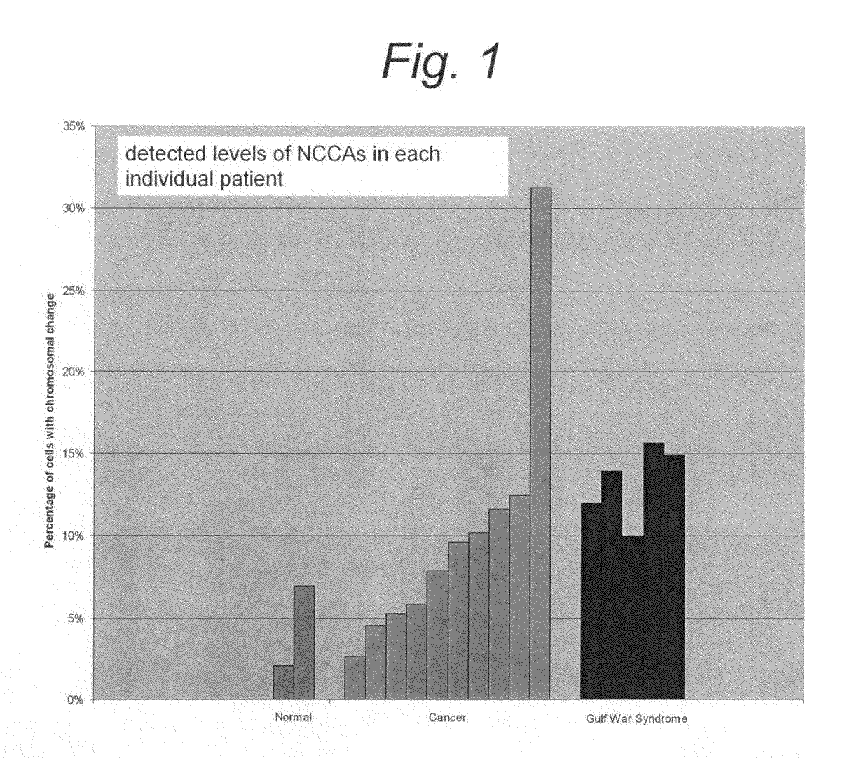

A mitosis-specific event. Chromosome fragmentation was observed at low levels (typically <5% of mitotic cells, depending on specific cell line) in a number of cell lines without any drug treatment. To show that chromosome fragmentation is generated directly from mitotic cells and not from cells in interphase, the population of mitotic cells was increased via shaking off nonadherent mitotic cells and treating with colcemid to arrest them in mitosis and concurrently treating them with the topoisomerase II inhibitor, doxorubicin. Nearly 100% of these mitotic cells showed a degree of chromosome fragmentation within 12 h of this treatment (FIG. 4D). When interphase cells were treated with doxorubicin and colcemid that was preceded by a double thymidine block, no chromosome fragmentation was observed for up to 24 h. BrdUrd incorporation was monitored during induction of chromosome fragmentation and no BrdUrd incorporation was observed (FIG. 1D), suggesting that despite the morphological similarity of chromosome fragmentation and PCC, they are in fact distinct processes.

Additional indications that chromosome fragmentation takes place exclusively in mitotic cells include assessment of the phosphorylation status of histone H3 at serine 10. Immunofluorescent staining of phosphorylated H3 (Ser10), revealed the majority of mitotic cells, fragmented or not, show positive H3 staining, suggesting that these fragments are indeed derived from condensed mitotic chromosomes (see, FIG. 3B). Multiple color spectral karyotyping was then performed to examine the relationship between fragmented and normal chromosomes as spectral karyotyping can precisely identify individual chromosomes. Results from spectral karyotyping analysis (see, FIG. 3C) show that even highly fragmented chromosomes are condensed and have a grouping and localization similar to what would be expected if that chromosome was intact.

Chromosome fragmentation results in cell death. Fragmented mitotic figures with extensive chromosomal damage would seemingly be incompatible with survival. To test the direct link between fragmentation and cell death, mitotic cells were treated to induce chromosome fragmentation and followed this with calcein AM and ethidium homodimer staining to assess viability. Following treatment, cells were collected and analyzed for viability and the frequency of chromosome fragmentation. It was found that chromosome fragmentation does result in cell death as chromosome fragmentation increases (see, FIG. 4A) as viability decreases (see, FIG. 4B). Additionally, cells were observed that displayed extensive ethidium homodimer labeling of fragmented chromosomes (see, FIG. 4C), further illustrating that chromosome fragmentation directly results in loss of membrane integrity and loss of viability. Therefore, chromosome fragmentation is a form mitotic cell death.

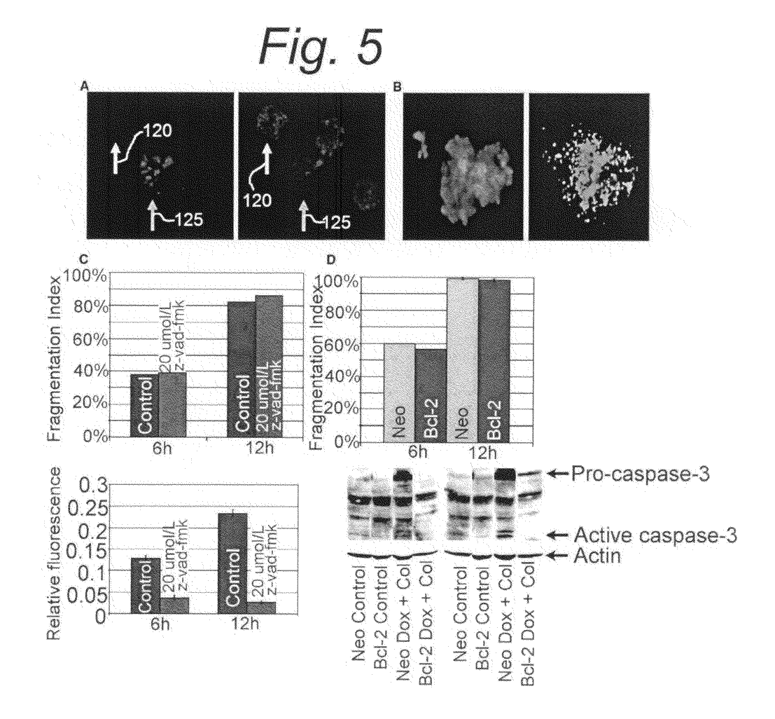

This process differs from typically described apoptosis. Next, a determination was made whether chromosome fragmentation is a typical apoptotic process. TUNEL staining performed on cells undergoing chromosome fragmentation shows negative staining of cells displaying chromosome fragmentation (see, FIG. 5A) in agreement with previous reports. Cells with typical apoptotic morphology (round clusters of DNA) from the same slides, however, show positive TUNEL staining (see, FIG. 5A) as did positive controls (data not shown). Also, apoptotic oligosomal DNA ladders were not evident after induction of fragmentation (data not shown). Chromosome fragmentation, however, involves strand breaks as evidenced by positive —H2AX staining along chromosomes (see, FIG. 5B). This has been implicated in apoptosis and damage repair signaling.

FIGS. 5A, 5B, 5C, and 5D are photographic representations that illustrate TUNEL staining performed on cells undergoing chromosome fragmentation to show negative staining of cells displaying chromosome fragmentation and to illustrate that chromosome fragmentation is not apoptotic. Fragmented chromosomes show negative TUNEL staining (FIG. 5A, arrows 120, DAPI right, TUNEL left). Typical apoptotic bodies display positive TUNEL results (arrows 125) as do positive controls (data not shown). Chromosome fragmentation results in intense —H2AX staining (DAPI left, FITC right), which is an indicator of strand breaks, indicating that despite negative TUNEL staining, there is indeed strand breakage (FIG. 5B). Caspase inhibition does not inhibit chromosome fragmentation (FIG. 5C). Cells pretreated with broad-spectrum caspase inhibitors and then treated to induce chromosome fragmentation in the continued presence of, or after removal of, caspase inhibitors show similar frequencies of chromosome fragmentation at 6 and 12 h of treatment (top). Notwithstanding that there was no change in the frequency of chromosome fragmentation, levels of caspase-3 activity significantly (6 h, P=0.003479; 12 h, P=0.00007) differed between the cells treated with z-vad-fmk and those without (bottom), indicating that activation of the initiator caspase, caspase-3, is not required for chromosome fragmentation. Interestingly, treated cells in the presence of caspase inhibitors tend to exhibit later stages of fragmentation compared with cells removed from caspase inhibition after 12 h of treatment, although total chromosome fragmentation frequencies remain similar. Chromosome fragmentation frequencies were unaltered in H460 cells that overexpress the apoptosis inhibitor Bcl-2 when compared with H460 cells carrying an empty neo vector when chromosome fragmentation is induced once again, indicating that caspase activation and/or mitochondrial membrane permeabilization are not required for chromosome fragmentation to occur (top). Levels of pro- and active caspase-3 are higher in the H-460 neo control compared with H460-Bcl-2-overexpressing cells when fragmentation is induced for 6 and 12 h, as indicated by Western blotting (bottom).

Mitotic cells were pretreated with the caspase inhibitor z-vad-fmk or with a negative control of z-vad-fmk for a short period then treated with colcemid and doxorubicin and analyzed for frequency of chromosome fragmentation. As shown in FIG. 5C (top), chromosome fragmentation frequencies were not significantly altered upon caspase inhibition. Furthermore, FIG. 5C (bottom) shows that caspase-3 reactivity is largely inhibited when 20 μmol/L zvad-fmk is added. However, levels of caspase-3 activation increase from 6 to 12 h of treatment in the uninhibited cells. Bcl-2 overexpression was also found not to inhibit chromosome fragmentation. Using two variants of the non-small cell lung cancer cell line, H460, the first overexpressing Bcl-2 and the second carrying an empty neo vector, chromosome fragmentation was induced. Both lines showed similar frequency of chromosome fragmentation as indicated in FIG. 5D (top), whereas levels of pro-caspase-3 and activated caspase-3 were increased in the treatments on cells containing the empty neo vectors and levels and activation were decreased or eliminated in cells overexpressing Bcl-2 (FIG. 5D, bottom). This indicates that unlike apoptosis, Bcl-2 overexpression does not influence frequency of chromosome fragmentation.

This process differs from typical mitotic catastrophe. If chromosome fragmentation lacks the classic hallmarks of apoptosis perhaps it was mitotic catastrophe. During treatment, cells in culture became large and detached as reported. Cytogenetic analysis coupled with phosphorylated H3 antibody staining indicated that the cells that were undergoing mitotic catastrophe may not be typical mitotic cells. Cells were harvested after 24, 48, and 72 hours and prepared for cytogenetic analysis or as described. Chromosome fragmentation was not observed, indicating that this model of mitotic catastrophe is distinct from chromosome fragmentation. Mitotic shake off was then performed on the HCT116 14-3-3−/− cell line and accumulated mitotic cells were treated to induce fragmentation. Interestingly, HCT116 14-3-3−/− seemed to be resistant to chromosome fragmentation, as very few mitotic figures displayed fragmentation, whereas the parental strain of HCT116 shows drastically increased frequency of fragmentation when the same treatment was applied.

A second model of mitotic catastrophe induced by aphidicolin treatment in p53 null HCT116 cells was assessed for chromosome fragmentation. A mitotic fraction of 3.2% at 72 h [compared with over 20% by three-dimensional fluorescence-activated cell sorting (FACS) analysis of DNA content and H3 phosphorylation] and 5.4% at 96 h was observed via the present conventional cytogenetic analysis. Chromosome breaks were evident in the majority of chromosome spreads at 96 h. These breaks in contrast to chromosome fragmentation were more regular in size. Most cells do not show extensive breaks as typical chromosome fragmentation does, and when viewed at ×20 magnification these mitotic spreads appear normal. In the case of chromosome fragmentation, normal mitotic spreads are typically discernible from chromosome fragmentation at ×20 magnification. A minority of mitotic cells show some chromosome fragmentation. However, even the total mitotic fraction does not represent the proportion of cells reported as undergoing “mitotic catastrophe.” Previous reports have failed to show mitotic specific proteins such as MPM2 or phosphorylated H3. Mitotic chromosomes with and without breaks stained intensely positive for phospho-H3 (Ser10); however, a number of cells with no apparent chromosomal condensation showed slight H3 staining indicating that those cells previously identified as mitotic by FACS analysis are not actually mitotic.

A true mitotic cell death: Chromosome fragmentation is an event that takes place in stressed cells and results in death directly from mitosis, seemingly unlike what has been termed mitotic catastrophe. Chromosome fragmentation seems to be a form of programmed cell death although the possibility remains that chromosome fragmentation is a form of necrosis. Chromosome fragmentation is not a form of apoptotic cell death, as it lacks the morphologic and biochemical markers of apoptosis.

The process of chromosome fragmentation results in cell death as evidenced by loss of viability in cells undergoing chromosome fragmentation (see, FIGS. 4A and 4B). Further, chromosome fragmentation cannot be induced from cells in other stages of the cell cycle. When cells were blocked in S phase with a double thymidine block, chromosome fragmentation was not apparent, although massive cell death occurred likely due to G2 arrest and subsequent apoptosis induced by the doxorubicin. Furthermore, the lack of BrdUrd incorporation in cells undergoing chromosome fragmentation shows that the chromosomes are not induced to condense during S-phase. Thus, chromosome fragmentation does not represent premature chromosome condensation during S-phase. Rather, chromosome fragmentation is a phenomenon that takes place during mitosis and occurs when chromosomes are damaged either by drug treatment or inappropriate passage of damaged cells through the G2 checkpoint. It should be noted that previous reports have shown spontaneous fragmentation in the ATR knockout model, most likely due to cells entering mitosis with DNA damage and ineffective G2 checkpoint activation. Therefore, chromosome fragmentation is not a unique phenotype of the ATR−/− genotypes, but rather the general feature of unstable genomes.

Based on the morphologic characterization, chromosome fragmentation might offer insight into the mechanism of the long-described phenomenon of chromosome pulverization. Frequently observed after viral infection and thought to occur from cell fusion, chromosome pulverization has often been linked to the prematurely condensed chromosomes and thus was thought of as occurring in interphase nuclei. The lack of BrdUrd incorporation noted in chromosome fragmentation (see, FIG. 3D) challenges this notion and shows that fragmentation is the likely mechanism of chromosome pulverization in cases of drug treatment or viral infection.

Nonapoptotic cell death: Chromosome fragmentation lacks many of the hallmark features of apoptosis, including the characteristic pattern of DNA fragmentations detectable by the TUNEL reaction. Caspase activation is also a hallmark of apoptosis. As caspase activation is intimately involved in apoptosis, the inventors herein sought to determine whether chromosome fragmentation was also dependent on caspase activity. These results were not due to cells already undergoing apoptosis as cells were rinsed to remove detached dying or mitotic cells and cultured for 15 h in the presence of the caspase inhibitor, shaken off, and then treated with colcemid and doxorubicin in the continued presence of z-vad-fmk or with z-vad-fmk removed. Although there was strong repression of caspase activation in the presence of z-vad-fmk (FIG. 5C, bottom), the fragmentation index was not significantly altered, indicating independence of caspase activation in chromosome fragmentation.

Bcl-2 is an antiapoptotic protein that is commonly overexpressed in tumors. Bcl-2 overexpression inhibits mitochondrial pore opening, cytochrome c release, and subsequent apoptosis. Bcl-2 overexpression was shown not to alter the frequency of chromosome fragmentation when it was induced by drug treatment of mitotic cells, despite the repression of caspase-3 expression and activation detectable in the Bcl-2-overexpressing cells compared with cells expressing an empty neo vector only. Chromosome fragmentation therefore lacks many of the major hallmarks of apoptosis, DNA strand breaks, sensitivity to caspase inhibition, and sensitivity to Bcl-2 overexpression. However, chromosome fragmentation does display double strand breaks as noted by positive —H2AX staining (see, FIG. 5B), which can serve as an early apoptotic sign.

The present mitotic cell death process differs from previously described mitotic catastrophe. Mitotic catastrophe has been loosely defined as cell death that results from aberrant mitoses. It has been contradictorily suggested to be apoptotic and nonapoptotic, but seems to typically be linked with segregation abnormalities. Not only do the definitions of mitotic catastrophe differ but there is also a lack of morphologic characterization of chromosomes from cells undergoing mitotic catastrophe. It has been strongly suggested that more descriptive terms for mitotic cell deaths be used to avoid the vagueness and confusion of the term “mitotic catastrophe.” Due to the confusion of mitotic catastrophe, and its apparent importance, chromosome fragmentation was assessed in two representative systems of mitotic catastrophe. Cells were induced to undergo mitotic catastrophe according to published reports. Half of the cells were prepared using reported protocols and half were prepared according to standard cytogenetic procedure to score chromosome fragmentation frequency.

One of the first models of mitotic catastrophe described in human cells is the HCT116 cell line without 14-3-3 function. Cells treated with a low dose of doxorubicin undergo mitotic catastrophe, which was described as a near-steady population of 2N cells, a declining population of 4N cells, and an increase in sub-2N content. Cells were subjected to the same treatment as reported collected and processed by conventional cytogenetic protocols but chromosome fragmentation was not evident although irregularly shaped small nuclei that seem to have condensed chromatin due to their uniform dark staining are quite regular. When cells were stained for phosphorylated H3, only a very small fraction of cells show any positive signal, leading to the conclusion that mitotic catastrophe described in such a system may not truly be mitotic.

There is no consensus on the exact role of 14-3-3 in mitotic catastrophe. On one hand, 14-3-3 is described as a regulator of the G2 checkpoint that functions with cdc25 to sequester cdc2 in the cytoplasm, whereas other reports suggest that 14-3-3 does not take part in the G2 checkpoint, but rather may serve as an antiapoptotic protein much like surviving. Nevertheless, the mitotic catastrophe shown by the HCT116 14-3-3−/− model is distinct from chromosome fragmentation.

It is reported that by 72 h of aphidicolin treatment in the HCT116 p53−/− system, more than 20% of cells were mitotic as determined 4N content and positive phospho-H3 staining, whereas 40% of the cells died. A similar number of mitotic figures upon cytogenetic analysis was not observed, although a portion of cells in interphase showed phosphorylation of histone H3 and may represent a portion of the mitotic population described by FACS analysis.

Mitotic catastrophe is commonly defined as cell death after abnormal or failed segregation. In fact, the mitotic catastrophe generally is not considered a form of cell death, but rather an irreversible trigger of cell death. Further, the Nomenclature Committee on Cell Death realizes the ambiguity of mitotic catastrophe and suggests use of descriptive terms such as “cell death at metaphase” and “cell death preceded by mutinucleation.” Neither model of mitotic catastrophe displays typical chromosome fragmentation in concordant levels to the portion of the cell population dying by mitotic catastrophe. This is likely due to chromosome fragmentation resulting in death directly during mitosis, whereas the majority of cell death due to mitotic catastrophe in the two systems examined takes place after the completion of an abnormal mitosis. Although the mechanistic differences between chromosome fragmentation and mitotic catastrophe are yet to be addressed in depth, importantly they are distinct based on the fact that one is a typical mitotic event and the other does not directly seem to be. Nevertheless, further study is warranted regarding the molecular links between mitotic catastrophe and chromosome fragmentation.

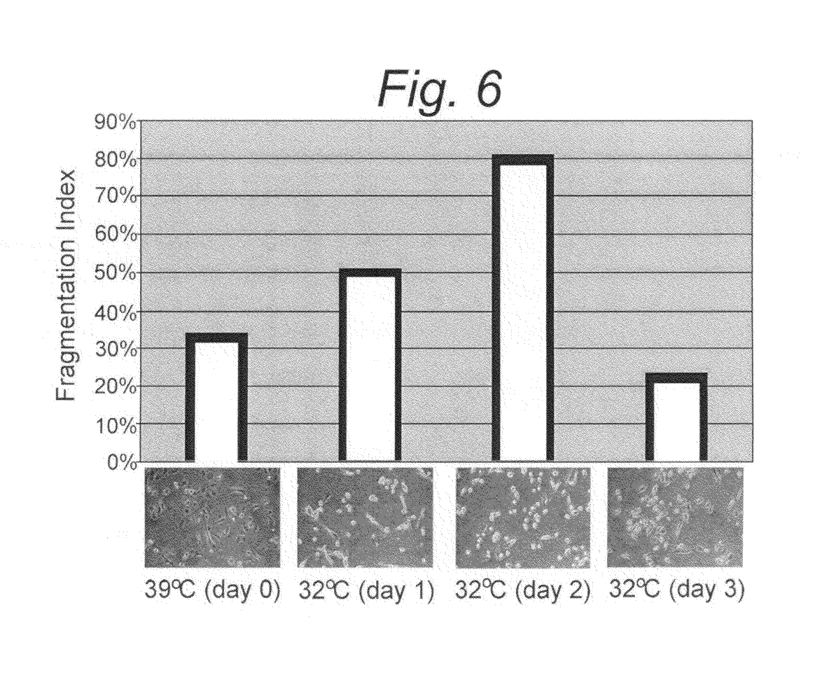

Link to genomic instability: Interestingly, it was observed that the frequencies of chromosome fragmentation correlate to levels of genomic instability of a given cell line. Elevated chromosome fragmentation was often detected from cell lines with high level of instability. Such correlation is nicely reflected by the dynamics of a given cell population in that cells are more likely to undergo chromosome fragmentation during the transition from periods of instability to stability. During cancer progression, there are distinct transitional stages of genomic stability as evident by patterns of clonal and nonclonal karyotypic progression. The cell line H1299 v-138 is a line that expresses a temperature-sensitive p53 mutation. This system is genomically unstable at the restrictive temperature (data not shown). Culturing cells at the restrictive temperature results in rapid growth and increased nonclonal chromosome aberrations (NCCA), indicating the presence of an unstable phase. When there is a shift to the permissive temperature, there is a major die off as the cells regain relative genomic stability. As the cells die off, death by chromosome fragmentation is elevated (FIG. 6), indicating that chromosome fragmentation is involved in the elimination of genomically unstable cells. When cells are continuously cultured at the same temperature, there is a dramatically decreased rate of chromosome fragmentation that is highlighted at the permissive temperature when p53 function is restored.

The transiently increased frequencies of chromosome fragmentation and NCCAs can be achieved by additional drug treatment or even by switching from the restrictive to permissive temperature, demonstrating that genomic instability and the mitotic cell killing are tightly linked possibly by evolutionary selection. When both internal and environmental conditions change (either by dysfunction of p53, or simply by switching the temperature or carcinogen treatment), genomic instability, reflected by increased NCCAs, will lead to cell death reflected by increased chromosome fragmentation frequency, which will in turn reduce the heterogeneity for a given cell population. According to this analysis, the frequency of chromosome fragmentation should be used to monitor the in vivo and in vitro response of chemotherapeutics. The high frequencies of fragmentation often occur after chemotherapy was observed.

FIG. 6 is a graphical representation that illustrates that the chromosome fragmentation rate is associated with genomic instability. The H1299 v-138 cell line contains a temperature-sensitive p53 mutation with the restrictive temperature being 39° C. and the restrictive temperature being 32° C. H1299 v-138 cells were grown at 39° C. and then shifted to 32° C. During the shift, the cells progressively detached and died over a period of 5 d until few viable cells remained in agreement with previous reports. Upon temperature shift, spontaneous chromosome fragmentation rates increased, then declined, as the number of dying cells and overall genomic instability decreased.

Paradoxically, the process of fragmentation could also introduce additional heterogeneity. This may occur through the differential elimination of certain chromosomes leading to aneuploidy, a form of NCCA that serves as the driving force of cancer progression. Mitotic figures in which only one or two chromosomes were fragmented (FIG. 3C) were frequently observed. These cells are expected to survive but display aneuploidy. In addition, if a cell is undergoing chromosome fragmentation and is not able to complete the process of death, these fragments can be lost and form micronuclei. These can be “stitched” together by various repair complexes, or can result in double minute chromosomes if they are retained and replicated. Thus, incomplete chromosome fragmentation can potentially lead to genomic instability, which, in turn, could lead to genome complexity that drives cancer progression.

It has herein been shown that during chromosome fragmentation, there is intense —H2AX staining along all chromosomes (FIG. 5B). Phosphorylation of H2AX is one of the initial events in the recruitment of repair complexes. If chromosome fragmentation is halted during the process, it is conceivable that repair complexes could be recruited, fragments rejoined, and karyotypically complex chromosomes could be formed. Thus, chromosome fragmentation is a clinically important phenomenon that can result in DNA damage, change in chromosome number, and changes in NCCA levels that increase population diversity. The degree of chromosome fragmentation could also serve as a measurement of induced and spontaneous mitotic death and transient genomic instability. For example, for some patients, chromosome fragmentation has been observed in peripheral lymphocytes even months after chemotherapy is withdrawn. High frequencies of fragmentation have also been observed from the blood of brain tumor patients without any chemotherapy. Obviously, determining the clinical significance of these observations requires more research.

The question herein raised is what is the clinical significance of chromosome fragmentation? First, all chemotherapeutics tested to this point can generate increased frequencies of chromosome fragmentation. For example, doxorubicin and methotrexate, which exert significantly different effects and cause death by different mechanisms, can induce chromosome fragmentation. This indicates that the initial pathway to trigger the cell death process could be different, but as long as the killing happens in mitotic cells, chromosome fragmentation is a common form of cell death. Second, limited clinical samples were also examined from lymphocyte cultures of cancer patients and chromosomal preparations of primary tumors. Elevated fragmentation is often detected particularly in individuals under chemotherapy (data not shown).

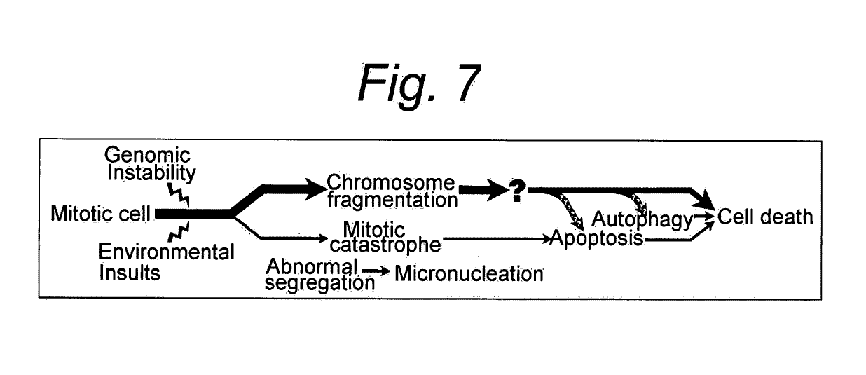

FIG. 7 is a representation of a model that illustrates the relationship between chromosome fragmentation and other types of cell death. Chromosome fragmentation differs from mitotic catastrophe in that cells undergoing chromosome fragmentation commit to death during mitosis, whereas cells dying via mitotic catastrophe die after mitosis.

Although the mechanism of the relationship of chromosome fragmentation with other forms of cell death requires further characterization, the inventors herein propose a model showing the interconnectivity of various forms of cell death (see, FIG. 7). It is possible that chromosome fragmentation represents progressive degradation of DNA from chromosomes to chromosome fragments to smaller DNA fragments or that it is an initial event that results in the formation of smaller chromosomal fragments that are easier for cells to further digest possibly via apoptotic pathways as the cell dies. An additional possibility is that chromosome fragmentation could represent a form of autophagy, as it has been shown that chromosomes can be enveloped by autophagic vesicles upon intense free radical formation. In fact, for the early fragmented mitotic figures, often one or a few chromosomes were fragmented indicating such a connection. Another challenge is to understand the molecular mechanism of chromosome fragmentation, which molecules are involved in digestion, and how they are activated when the genome is unstable or when the cell is insulted.

### Conclusions Regarding Chromosome Fragmentation

Chromosome fragmentation is a clinically relevant mode of mitotic cell death that results in the progressive degradation of chromosomes during mitosis. Chromosome fragmentation is apparently not apoptotic and differs from models of mitotic catastrophe. Chromosome fragmentation is intimately linked with genomic instability, serving to remove cells that display an altered genomic system stability, which is commonly seen in cancer progression. Furthermore, as it represents one form of NCCA, chromosome fragmentation can lead to increased genome stability if the process is compromised, which could lead to the increased genome complexity noted in cancer. This form of cell death needs to be monitored especially in cancer where genome stability plays such a key role in disease progression. Thus, chromosome fragmentation is a new form of mitotic cell death that is of great importance in the treatment of cancer.

Formulation of a Model that Illustrates the Relationship Between Evolutionary Concept and Molecular Mechanisms

Summarizing of all models that have been analyzed, it is clear that in each case examined (a given experimental model based on a selected cell line, individual animal lesion), a specific or combination of specific molecular pathways can be illustrated and thus linked by molecular analysis. However, there is no common molecular basis or mechanism leading to cancer evolution in general, since no specific form of genomic aberration is universally shared among diverse cancer cases. This is also true at the sequence level, as a recent large scale sequencing project indicated that there are many different genetic combinations or hills at the gene level in the context of the evolutionary adaptive landscape. If one abstracts from these seemingly specific and unrelated causes, including a number of known molecular pathways, elevated DMFs, increased ploidy, simple or complex chromosomal translocations, and large scale stochastic changes at the gene level and epigenetic level, the picture of a common mechanism will emerge. That mechanism is karyotypic heterogeneity rather than a specific molecular pathway.

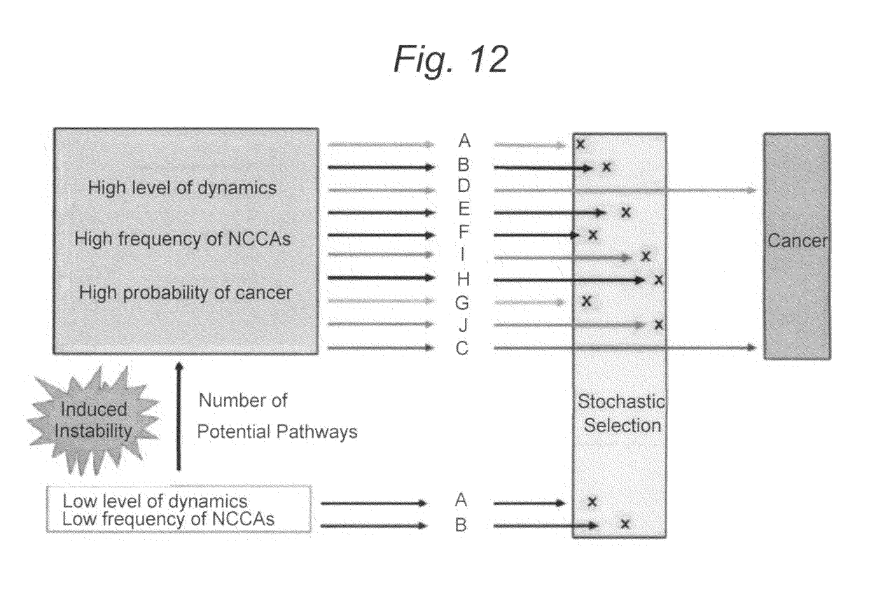

FIG. 12 illustrates the evolutionary mechanism of cancer and its relationship with molecular mechanisms. The present evolutionary explanation of why there is a correlation between elevated NCCAs, genome diversity and tumorigenicity is illustrated in the model shown in FIG. 12. The evolutionary mechanism of cancer formation is summarized as three key components: 1) system instability; 2) increased system dynamics or population heterogeneity (reflected as an increased probability of a hit of a specific pathway or potential pathways); and 3), natural selection at the somatic cell level. There are many different molecular pathways that can trigger system instability, and it is the unstable system that activates different molecular pathways as the response to system instability. The somatic selection process stochastically favors different packages of genome alterations. The lower left box in the figure represents a normal stable state that typically generates infrequent NCCAs and when they do occur will likely go extinct.

With increased instability, much higher levels of NCCAs occur representing an increasing number of potential genome systems coupled with specific molecular pathways. Each array represents a given molecular pathway, or the so called molecular mechanism. The increased number of pathways (represented by various colored arrows) increases the probability that evolution will proceed at a faster rate progressing much further in selected cell populations with some eventually achieving cancer status (the evolutionary mechanism).

Based on the concept of cancer evolution and the realization that cancer is a disease of probability, one can understand why elevated genome diversity will lead to the success of cancer evolution regardless of which molecular pathways or mechanisms are involved. This diagram links various molecular mechanisms with the evolutionary mechanism of cancer. It not only can explain the knowledge gaps between basic experiments and clinical findings (in experimental systems, many cancer genes can effectively cause a cancer phenotype, yet, these gene mutations only account for a small portion of the clinical cancer cases), but also focuses attention on the evolutionary mechanism rather than molecular mechanisms. There are large numbers of different molecular mechanisms that for all practical purposes cannot be predicted, in contrast, it would be much more useful to predict the increasing probability of cancer using the evolutionary mechanism. Such relationship between evolutionary mechanism and molecular mechanisms of cancer can simply be stated as follows:

Evolutionary Mechanism=εIndividual Molecular Mechanisms

This formula offers insight into the relationship between system instability, karyotypic heterogeneity, individual molecular mechanisms and tumorigenicity.

As illustrated by the present inventive model of FIG. 12, the linkage between the elevated degree of NCCAs and tumorigenicity explains the mechanism of cancer in simple evolutionary terms. A stable cell population, with lower degrees of change, translates into a lower probability of cancer formation. Increased system instability, in contrast, results in an increased probability of cancer formation. The present experimental data illustrate the evolutionary mechanism of cancer formation and that system instability is the key causative factor. As previously noted, many genetic, metabolic and environmental elements can contribute to genome system instability, including system dynamics.

When unstable, the genome system offers a higher probability of change or diversity, reflected as variable karyotypes that offer a greater number of different molecular pathways, which are the material for evolutionary selection as well as a precondition to establish new genome systems.

The seven examples described above involved both human and mouse cells of different cancer types and the malignant phenotypes have been linked to specific but different precipitating events. These events range from increased microsatellite instability and allelic loss, to chromosome ploidy, different chromosomal translocations and numerical aberrations, to HOXA1 gene and c-Myc expression, and to down-regulation of E-cadherin, as well as centrosome amplification caused by Rad6 and stromal-epithelial interaction (see, Table 3). For each characterized system, the linkage between a specific pathway or genetic event has been described as a given molecular mechanism. When considering all systems together, however, none of these events can be used to explain all cases. Significantly, the only common link to tumorigenicity is increased levels of NCCAs. Clearly, the present correlative observation between increased levels of NCCAs and tumorigenicity supports the causal relationship between system instability reflected by elevated NCCA levels and tumorigenicity. Thus, such a correlation offers an evolutionary mechanism for cancer formation by generating cellular diversity.

TABLE 3
Various molegular mechanisms are linked to the increase in NCCAs,
the common feature of the evolutionary mechanism of cancer

| | Previous findings (molecular | |
|---|---|---|
| | mechanisms: features or | |
| Cell model | identified pathways | Current common findings |
| LNCaP | Increased microsatellite instability; | Increased frequencies |
| | gradually lost androgen response; | of NCCAs; increased |
| | Increased tumorigenicity | genome diversity |
| MCF10DCIS.com | Stromal-epithelial interaction; | Increased frequencies |
| | increasingly invasive phenotypes | of NCCAs; increased |
| | | genome diversity |
| MCF10-CSC | Increased ration of BCL-xL/Bax; | Increased frequencies |
| | increased expression of PCNA, | of NCCAs ploidy; |
| | gadd45; increased tumorigenicity | increased |
| | | tumorigenicity in vivo |
| MCF10-HoxA1 | Activation of cdD1 and Bcl-2; | Increased frequencies |
| | increased tumorigenicity | of MDFs; increased |
| | | genome diversity |
| Mouse Ovarian | Change: cytoskeleton and focal | Increased frequencies |
| | adhesion complex, down: | of NCCAs; increased |
| | E-cadherin and connexin-43, | genome diversity |
| | increased tumorigenicity | |
| MCF10-Rad6 | Centrosome amplification, | Low level of structural |
| | aneuploidy and transformation; | NCCAs; aneuploidy |
| | benign hyperplastic lesions | |
| Myc-transgenic mice | Expression of A2 and E2F1; | Increased frequencies of NCCAs |
| | increased tumorigenicity | |

It should be pointed out that the context of the term “mechanism” is very different among academic fields. In molecular biology, for example, mechanism typically refers to a change in a molecule that results in a specific phenotype or other molecular events. The evolutionary meaning of mechanism refers to the generation of cellular heterogeneity, which is the instrument or means of natural selection through population diversity. The evolutionary mechanism is therefore much broader than the molecular mechanism and can be achieved by many different molecular mechanisms or other mechanisms under specific circumstances. For example, different types of stress can trigger system instability. In molecular terms, the stress can be classified into specific molecular actions such as ER stress, metabolic stress, stress resulting from ineffective DNA repair, over-expression of certain oncogenes, etc.

Irrespective of the type of molecular stress, the system response is not stress specific but displays a common response increasing the level of system dynamics, confirmed by the elevation of NCCAs. Despite the common response of elevated NCCAs, a specific NCCA (or number of NCCAs) will be selected, however the associated molecular pathways will be more or less unpredictable and will continuously change. Each molecular mechanism that generates stress and the response to stress can contribute to or is even equal to the evolutionary mechanism of each specific case. However, the general evolutionary mechanism cannot be sufficiently explained or predicted by individual molecular mechanisms as there is no shared molecular mechanism in all cancer cases. Similarly, the term causative relationship has a different meaning when considering the difference between a single molecular pathway and a complex system.

In the molecular sense, the causative relationship is defined within an isolated network where molecule A or event A (called cause) leads to B (called effect). In a complex system, however, cause and effect relationships might not be so narrowly defined nor maintain the same meaning as illustrated by experiments. An experimentally defined relationship setup between two parties can be easily changed when additional interactions are included. In fact, complicated interactions are always present in natural settings but are ignored in experimental analyses.

To analyze complex systems, correlation studies are thus fundamentally important as causative studies among lower level parts of a system in an isolated setting may not be as reliable in the context of a complex system. In contrast, to study the mechanism of cancer evolution (and not individual molecular mechanisms), a general correlative relationship where system instability results in population diversity, and the population diversity provides the necessary pre-condition for cancer evolution to proceed, in fact illustrates the causative relationship between system dynamics and cancer.

It is likely that many different pathways are stochastically involved and selected when there is elevated instability and genetic diversity, based on the stochastic nature of karyotypic aberrations and the mechanism of cancer evolution. For example, some NCCAs may activate dominant oncogene defined pathways, while others may have various combinations of minor changes that eventually result in the final phenotypes of uncontrolled growth. The link between NCCAs and tumorigenicity in the majority of cancers supports the present model.

The concept herein disclosed predicts that the result of genomic instability (inherited or induced) is the generation of population diversity (evident though clonal diversity or non-clonal diversity or the combination of both) which drives the cancer evolutionary process. The cases analyzed here represent the tip of the iceberg, as the often hidden link between population diversity and tumorigenicity can be easily found in cancer literature. Although most of these reports focus on specific molecular pathways, including specific oncogenes, tumor suppressor genes, epigenetic regulation, or genes responsible for tissue architecture, most of these aberrations can be linked to overall genome instability resulting in population diversity. This fits well with the genome-centric concept of cancer.

### Advantages of Using NCCAs/CCAs to Monitor the Cancer Evolutionary Process

Initially demonstrated in the in vitro immortalization model, the high level of NCCAs and dynamic interaction between NCCAs and CCAs plays an important role in cellular immortalization. The current study further provides solid evidence that elevated NCCAs are directly linked to tumorigenicity.

Most recent studies have focused on tracing specific gene mutations or methylation patterns due to the available technologies. However, there are some serious limitations regarding the strategies of gene based evolutionary analysis. First, the current technologies used in genetic analyses are based on a mixture of cell populations that only artificially profiles the most dominant clonal population and ignores the importance of heterogeneity. Second, as illustrated in previous publications, most solid cancers involve progression with high levels of stochastic change, where it is difficult to trace the genetic changes, and only during slow phases (prior to the blastic phase in CML, for example) of limited blood based cancers or solid tumors are some genetic changes traceable.

Even in blood cancers, it is almost impossible to trace genetic changes in late stages. In addition, according to the theory of orderly heterogeneity and system complexity, it might be more meaningful to trace the higher levels of genetic organization (genome) than the lower gene levels. More importantly, in somatic evolution, macro-evolution is the main mechanism and replacement of various genomes is the driving force of somatic cell evolution. When the genome context changes, even when the gene state is the same, it often does not keep the same biological meaning. For example, in different human pancreatic cancer cell lines, the K-ras gene mutation was linked to very different pathways, possibly due to the different context of genomes. Interestingly, NCCAs and epigenetic programming responding to stimulation of the Ras-MAPK pathway may be a better marker for cancer progression than the upstream mutated oncogenes. Therefore, by focusing on genome diversity, the overall evolutionary potential can be measured based on the karyotypic heterogeneity. Indeed, monitoring the karyotypic level is more effective than monitoring the gene level, as focusing on karyotypic heterogeneity is in fact studying the evolutionary mechanism while focusing on individual genes is studying a single specific molecular mechanism. Thus, the present study offers a new direction that uses the degree of karyotypic heterogeneity to effectively monitor tumorigenicity.

One issue that needs further analysis is the contribution of specific CCAs in combination with elevated NCCAs. Traditionally, attention has focused on CCAs as only clonal expansion was thought to be important for the accumulation of additional gene mutations. Genome dynamics drive cancer evolution, therefore it would be interesting to study how key CCAs play a role in increasing the population diversity rather than just providing proliferation. In agreement with the previous findings of the inventors, the current studies favor NCCAs rather than specific CCAs in monitoring genome system variance. However, it is still possible that for specific cases certain CCAs can contribute more to cancer evolution than others. For example, the mutation of p53, which can have many different functions, could be an example of a CCA that increases evolutionary dynamics, in addition to other functions.

It is thus possible that some powerful CCAs when combined with a certain level of NCCAs, would be most effective in terms of cancer evolution. In fact, consistent with previous publications, the inventors have observed that increased frequencies of complex CCAs (involving multiple translocations within one chromosome) are most frequently detected during the late stage of immortalization and during the formation of drug resistance.

It should be pointed out that, using a system approach to monitor NCCA/CCA dynamics is not contradictory to studying the function of various cancer genes, similar to not seeing the forest for the trees, these two approaches focus on two levels of genetic organization, and try to address different mechanisms (evolutionary and molecular) of cancer formation. Following decades of effort attempting to understand each molecular mechanism (including oncogenes, tumor suppressor genes, DNA repair genes, genes regulating transcription/RNA splicing/translation/protein modification and protein degradation, genes controlling cell cycle, cell death, cell proliferation and differentiation, cell communication as well as aneuploidy, micro-environments, and immuno-system responses), it seems that the complexity of cancer is too high and that just tracing individual pathways will not lead to understanding the nature of cancer due to the highly dynamic (stochastic and less predictable) features of this disease. There is a need to focus on the system's behavior and its patterns of evolution rather than focusing principally on individual pathways alone. Studying the dynamics of NCCAs/CCAs is just one such example of this approach.

### Some Technical Clarifications of Using NCCAs

The terminology non-clonal aberration is commonly used in the field of cancer cytogenetics. There seems to be no disagreement on the use of this term, but there is a distinct disagreement on their biological significance. The general rule in tumor cytogenetics has been that only clonal chromosomal abnormalities found in tumors were considered significant and should be reported.

A clone is defined as a cell population derived from a single progenitor. It is common practice to infer a clonal origin when a number of cells have the same or closely related abnormal chromosome complements. In practice, there are two meanings when the clonal aberration is used in cancer cytogenetic. First, it means that they are derived from a common ancestor within a defined time frame; and it also means that they are karyotypically identical or similar to each other. This latter meaning is of importance to cancer research, as technically speaking, all different cancer cells as well as normal cells of one individual must come from a single progenitor cell of a fertilized egg. However, different tumor cells and normal cells of one individual are not considered clones when they display drastically different genetic profiles (only when they share the same marker of abnormal chromosomes). The term “non-clonal” here is used to distinguish the clonal karyotypes rather than refer to cells not derived from a common ancestor.

Another note of caution is that whether or not an aberration is clonal depends on the time frame of examination and the level at which the study takes place (karyotypic vs. gene). Within a given period, the clonal aberrations can further evolve making it hard to realize that they are derived from a common ancestor. In addition, the concept of clonality can be applied to different levels of genetic organization. Cell populations with the same p53−/− mutation can be referred to as clonal at a specific locus, but they might be considered non-clonal at the karyotypic level.

To establish a precise scoring system to monitor the level of genome instability is challenging, as there are many different types of genome level alterations. By comparing the type and distribution of aberration frequencies for these model lines, it appears that the proportion of structural NCCAs represents the best biomarker. When NCCAs are used to score the level of heterogeneity, the total frequency of structural and numerical NCCAs should all be included. The structural NCCAs seem to play a more dominant role than numerical NCCAs, at least for the late stage of cancer progression (after transformation) that has been examined in this study. Chromosomal number variation plays an important role prior to the formation of structural NCCAs during the immortalization process of the mouse ovarian model.

The 4% cutoff of clonal/non-clonal is based on the standard of practice in medical genetics. It would be ideal if more than 100 mitotic figures could be included in the analysis and thereby use 1% as the cutoff line, but this is very time consuming and costly. In fact, a 4% cutoff is also reasonable as illustrated by studies conducted by the inventors herein with large numbers of cell lines and clinical samples.

For example, when studying the level of genome variations during the in vitro immortalization process, two additional cutoff lines were used (1% and 10%), the overall patterns of punctuated and stepwise phases of karyotypic evolution were the same as the 4% cutoff line (when the genome is unstable, the level of NCCAs often reaches over 20-50%). In the present immortalization model, when the cell population reached the unstable phase, NCCA levels were 100%, regardless of which cut off line was used to separate CCAs and NCCAs. In normal lymphocytes (based on both human and mouse data), the level of structural NCCAs is very low, in the range of 0.1-2%. For the purpose of establishing a baseline of structural and numerical NCCAs in normal individuals, over 100 mitotic figures are often scored. Interestingly, as illustrated by a current study, the differential frequency of NCCAs is more important than the absolute level of NCCAs as for each system tested, there seems to be a baseline of instability. No matter which cutoff line is used; the elevated NCCAs can easily be scored.

A key point here is the use of NCCAs rather than a given CCA to measure the overall system status and determine how stable a genome system is within a population. The population behavior or stability can be monitored by the degree of population diversity. It is believed that a new direction in cancer research will focus on controlling the process of system evolution, rather than focusing on specific drug targets, as there is no fixed target and just focusing on specific targets does not solve the issue of drug resistance in a dynamic evolving system. During the evolutionary selection process, any given pathway or specific target could become insignificant. Therefore, the apparent disadvantage of monitoring NCCAs in fact is an advantage in terms of monitoring the system status and its usefulness for system control.

One additional point needs to be clarified, the NCCA/CCA cycles reference herein could be described as clonal expansion and heterogeneity. The waves of dominant NCCAs or specific CCAs reflect the overall status of the stability of a population and the pattern of evolutionary dynamics. In contrast, using clonal expansion and genetic diversity to describe these two phases of population dynamics is not accurate. For example, during the clonal expansion phase, there is clearly genetic diversity. While, during the genetically diverse phase, all the new clones are still generated by clonal expansion. One of the key findings of the present karyotypic evolutionary study is that there are two typical types of clonal expansion illustrated by the immortalization model: clonal expansion with a lower level of system instability where expanded clonals share the majority of karyotypic characteristics of the parental cells; and clonal expansion with high levels of system instability where expanded clonals share few or no key karyotypic characteristics.

Interestingly, by just using a molecular profile such as tracing specific loci using a mixed cell population, drastically different evolutionary phases would not be appreciated. The partial reason that previous cytogenetic studies found the term clonal expansion and genetic diversity accurate is that the contribution of high levels of NCCAs were disregarded, resulting in easily identified marker chromosomes. From a molecular standpoint, it is easier to use the term clonal expansion in the molecular sense to study specific loci. When a specific locus is not an expansion, it can be called genetic diversity. However, if large numbers of loci were simultaneously monitored, it would be challenging to define the phase of clonal expansion. This is the exact situation when one studies karyotypic evolution based on a single cell within a dynamic cell population. In conclusion, it is useful to describe the change in frequency of the NCCAs or the amount of genetic diversity and also the phenomena of clonal expansion indicated by the types and frequency of CCAs.

By directly examining the tumors from mice, elevated NCCAs were detected in tumors but not normal tissues (tumor NCCA frequencies were at least 10-15 times higher). The inventors herein have conducted a clinical test on 10 normal and 10 prostate cancer patients. Using short term cultured lymphocytes, chromosomes were prepared and NCCAs were scored following spectral karyotyping. The average frequency of NCCAs was 0.9% in normal controls, and the average frequency of NCCAs in prostate cancer patients was 10.14% (over 11 times higher). The difference is highly statistically significant. This study represented an excellent example of using prostate cancer patient's blood to measure the overall system instability that indicates the cancer status of the solid tumor. At first glance, it is rather surprising as blood cells are not direct tumor samples. However, as cancer is a system disease, the linkage between the cancer status and the individual's overall instability must exist as illustrated by the present research. In fact, it is now well known that patients with solid tumors also display shortened telomeres when examining their blood cells.

For patients who quickly develop secondary cancers, high levels of NCCAs were detected from their cultured lymphocytes. For example, a young patient who developed a soft tissue tumor two years following successful chemotherapy of his blood cancer, the frequencies of NCCAs was 35% among examined mitotic figures. In normal individuals, the frequency of NCCAs is below 1-2%. The elevated frequency of NCCAs detected from this individual clearly demonstrated that a high level of genome instability is the underlying mechanism of the secondary tumor formation. This type of study sets up examples of monitoring chemo-treatment effects. This information is essential for treatment management in the future.

It is to be noted that the inventors herein have expanded the linkage between elevated NCCAs to genome system instability to other types of complex diseases in addition to cancer. The presented data on Gulf War Illness is just such an example.

For many complex diseases, the genetic contributing factors can be diverse, as different patients with the same type of disease do not share the same genetic contributing factors. Traditionally, the generally accepted concept has been that common diseases are caused by common genetic loci or are a result of common causes. Evidence generated by the inventors herein points to a new explanation for the causes of common diseases, such as Hypertension and Autism, each of these distinct causes are real and are very diverse, as many rare genetic variants are actually responsible for only a very small number of patients. However, many different factors can contribute to similar symptoms. This situation could well be applied to Gulf War Illness. Importantly, if the genome system is unstable, there could be a variety of symptoms, as many biological pathways could be stochastically impacted, leading to patients displaying different symptoms. Therefore, an entirely new approach is necessary to study these complex diseases by monitoring the level of genome instability, as clearly, diverse disease conditions can be influenced by varying war conditions with the environment playing an important role in this complex disease.

### Potential Clinical Implications

With an emphasis on the overall instability of the genome generating clonal diversity of cell populations as a major cause of cancer, this study favors a new approach to cancer research by focusing on the mechanism of cancer evolution rather than focusing on a specific molecular mechanism such as gene mutations or pathway. For the majority of cancer cases that involve multiple cycles of NCCA/CCA interaction, one specific pathway will likely not be successful. Thus more potential available pathways represented by high levels of NCCAs are necessary to develop a successful combination. It is likely that certain CCAs coupled with relatively powerful pathways can speed up the process of cancer evolution by drastically destabilizing the genome or by producing a high level of cell proliferation (such as specific powerful fusion gene mediated tumorigenesis). To complete the entire process of cancer formation, however, an overall high level of diversity is the key. Coupled with elevated levels of population diversity, there could be many pathways or great numbers of combinations of pathways that could lead to cancer through multiple steps.

The combination of dominant pathways and high level genome dynamics create the most favorable conditions for cancer evolution. Therefore, reduction of factors leading to genome instability and reducing cell population diversity should become new areas of focus for clinical research. For example, the key to cancer prevention and treatment is stabilization of the genome system. When genomes are unstable, blocking one particular aberrant pathway will likely not be successful, as new pathways will eventually emerge.

It is true that stochastic gene mutations also contribute to population diversity and can be traced in evolutionary studies. Similarly, epigenetic dynamics, as well as copy number variation all contribute to genome level alterations. It is very important to incorporate the degree of diversity at various levels. The hypothesis that using the frequencies of NCCAs might be inclusive of most of the other types of genetic and epigenetic dynamics seems to be correct and needs to be explored further, as the vast majority of other levels of genetic alterations will lead to karyotypic changes if system evolution occurs. Based on the viewpoint that the karyotype defines a genome system (both the overall expression pattern and the identity of a species), and that cancer evolution is driven by karyotypic mediated macro-evolution, it is anticipated that most cancer cases will have variable karyotypes. In fact, for many cases of leukemia, the seemingly normal karyotypes are only detected during the relatively stable phase of cancer progression. In the blastic phase, for example, karyotypic dynamics are overwhelming. Based on this consideration, this might be an advantage of using the highest level of genetic organization (the genome) to monitor genome system instability and evolution.

It is noteworthy that increased karyotypic diversity associated with various stages of cancer progression has been previously noted by others. The high level of karyotypic heterogeneity of NIH 3T3 cells has been linked to population diversity and transformation. The literature has also provided ample evidence to support this viewpoint, though the evidence has been largely ignored. For example, many genes or pathways that are linked to genomic instability in fact generate increased karyotypic diversity.

Interestingly, the link between population diversity and tumorigenicity reconciles the gap between certain experimental findings and clinical data when considering how these powerful oncogenes contribute to cancer. Under experimental conditions, most oncogenes are capable of inducing tumors, as the conditions have been created that increase the probability of cancer progression by using strong promoters and artificial selection. In real clinical cases, these well characterized oncogenes have limited involvement. The combination of strong oncogenes and tumor suppressor genes can significantly increase the probability of cancer progression under experimental conditions further demonstrating the importance of diversity as over or under expression of many oncogenes and tumor suppressor genes are directly or indirectly caused by genome instability.

Lastly, the present approach to monitoring genome diversity constitutes a valuable concept to develop assays for clinical use. It is known that the lesions in Barrett's esophagus exhibit the unique feature of stasis that allows the establishment of a correlation between stages associated with some key genes (one of the possible reasons is that the pre-cancer phase could be relatively more stable where there are more opportunities for clonal expansion). However, in contrast to Barrett's esophagus, most fast growing tumors exhibit high levels of diversity and dynamic karyotypic evolution, which is more typical of most progressive genomically unstable tumors. Monitoring of the levels of non-recurrent genomic aberrations in these latter types of tumors rather than using the degree of clonal aberrations is a more accurate level of genomic instability and is a practical method of accessing the likelihood of cancer progression. In addition to the potential benefit of using the level of NCCAs to monitor cancer progression and to provide needed tools for early diagnosis, this concept will help us to refocus on overall genomic instability and the generation of population diversity, rather than continue to focus entirely on specific pathways alone.

A New Concept Advocating Monitoring Genome Level Alterations and Suggested Methods of Implementing the Concept in a Clinical Setting.

Cancer genome sequencing has been proposed to identify common cancer genes, based on the assumption that cancer heterogeneity among patients is genetic “noise” and can be eliminated by validation using large patient samples. Unfortunately, this highly anticipated approach is delivering unwanted results. In yet another example of heterogeneity, the vast majority of gene mutations are not shared among patients.

Based on a genome theory of cancer evolution, and in accordance with the present invention, the key to understanding cancer is to not focus on specific genetic or epigenetic alterations, but to study the evolutionary mechanisms of cancer, and to effectively address the issue of genome system heterogeneity. Since cancer progression is characterized as genome mediated macro-evolution (rather than microevolution, or a developmental process), it requires a change of research strategy. Most of the molecular analyses of cancer have been focused on a molecule of interest, without considering the overall status of the genome system. It has been generally assumed that during molecular manipulation or specific targeting that the bio-system remains the same. This assumption has been pushed to the extreme where genome level information has become largely ignored by most of the molecular analyses. The fact is, however, when the overall karyotype changes, the role of the same gene may also be altered, as the function of genes are dependent on their genetic network, which is defined by the genome context.

Few studies have been carried out to analyze the biological meaning of drastic genome level alterations, which in fact could explain many contradictory findings occurring at the pathway level when cells with different karyotypes are analyzed. For example, the p53 pathway has been linked to diverse molecular mechanisms or pathways and at least 50 different enzymes can covalently modify p53 to alter its function, and several thousand genes have been shown to be directly regulated by p53. Each of these characterized functions represents one possible potential function defined by the genome context, including epigenetic regulation of the same genome but different tissue type. Clearly, for a given cell, most of the known mechanisms of p53 mutation cannot simultaneously function. One of the reasons the functional list of p53 mutations keeps growing is this molecule has been extensively examined using drastically different genome systems. As the majority of different cell lines and tumor samples that have been used in different experiments display different karyotypes, the large array of different p53 functions and its pattern within disease network in fact reflect the possibilities of functions created through system heterogeneity (in addition to the network complexity of a given system).

The level of the system that needs to become the primary focus: It is generally accepted that any bio-system can be classified into different levels and it is up to the individual researcher to choose the proper level of analysis based on available concepts and methodologies. According to the concept of complexity, the approach and rationale of reducing complexity to the lowest level usually does not work. Emergent properties of lower level parts are often very different from the overall system. Equally important is that information theory suggests that the selection of the level that controls a system is very crucial, and in contrast the level that is easiest to access information from is usually not very useful to control a system. Clearly, understanding how complexity and information theories apply to bio-systems will influence research strategies. It is known that research can apply at the nucleotide level, the gene and sub-gene level, multiple epigenetic levels and the genome level respectively. It is unknown however, which level is most important to the control of cancer and which should be the platform of current research.

The genome-centric concept of cancer evolution: genome level heterogeneity is a key to cancer evolution: At first glance, it seems rather complicated to deal with the issue of heterogeneity, as many levels and various types of heterogeneity are involved and the selection of a dominant pathway occurs stochastically. Paradoxically, despite the difficulties in establishing a causative relationship among individual molecular mechanisms within a complex bio-system, it is relatively easy to establish a causative relationship between system heterogeneity and cancer evolution, as heterogeneity is the necessary pre-condition needed for cancer evolution to occur, and the degree of heterogeneity can be measured. For example, if one focuses on the overall level of system dynamics, and if this is the principle level of selection during cancer evolution, then it is possible to monitor the overall pattern of heterogeneity at the principle level of evolution.

Considering that regardless of what factors induce system instability, increased levels of system dynamics can be measured using pattern changes such as in increased “random motion”. Thus, it would be more useful to study system behavior by monitoring the heterogeneity status rather than monitoring specific pathways, as it would be difficult to predict system evolution based on a specific pathway particularly when pathways have low penetration and low predictability.

Research conducted by the inventors herein illustrates the importance of using non-specific changes at the genome level to monitor genetic heterogeneity and its crucial role in cancer evolution, as the increased probability of cancer evolution becomes far more important than any specific pathway. Specifically, the inventors have defined cancer progression as macro-evolution where the major underlying force is karyotypic heterogeneity even though this process is associated with large numbers of seemingly random gene mutations and epigenetic alterations. Only within relatively stable stages where there is no karyotypic change, do gene mutations and epigenetic regulation play a dominant role (similar to the adaptation phase of micro-evolution). An illustration of this difference requires monitoring heterogeneity at different levels of genetic organization. To detect internal system modifications, monitoring gene mutations and epigenetic changes is more appropriate. From a system point of view, significant karyotypic changes represent a ‘point of no return’ in system evolution, even though certain gene mutations and most likely epigenetic changes can influence karyotypic changes

Measuring cancer heterogeneity at the gene, epigenetic and genome levels: Following the present demonstration of the importance of genome system heterogeneity in cancer evolution, a systematic analysis is needed to compare the different types of genetic and epigenetic heterogeneity and to incorporate them in an overall cancer evolutionary model.

a. The relationship between epigenetic change, gene mutation and genome alteration: Karyotypic evolution (system replacement) occurs during macro-evolution, while gene mutations are mainly linked to micro-evolution. Due to the involvement of global gene regulation (chromatin remodeling and genetic network regulation) and reversible features, epigenetic alteration can be considered as the prototype of genetic alterations and in particular genome level alterations. Therefore, it is likely that epigenetic alteration is an initial response when the genome system is under stress, which provides an increased probability for evolution dynamics to occur within a given genome context. When changes are selected by the evolutionary process, these changes can be fixed either at a specific gene level or at the genome level (achieving the transition from epigenetic to genetic changes).

Interestingly, a new run of epigenetic alteration can occur from newly changed genome topology (the genome defines the epigenetic potential). Therefore, the same epigenetic changes might have different biological meaning when occurring within different genome systems. For example, the hypomethylation of DNA can have unpredictable effects in terms of promoting or inhibiting cancer formation. As for the gene-genome relationship, any genome alteration will generate high levels of gene expression changes, some gene mutations that involve genome integrity can contribute to genome alteration, but only the genome level changes define a new system. In other words, karyotypic changes are the point of no return for systems and both gene mutation and epigenetic alteration can contribute to this process.

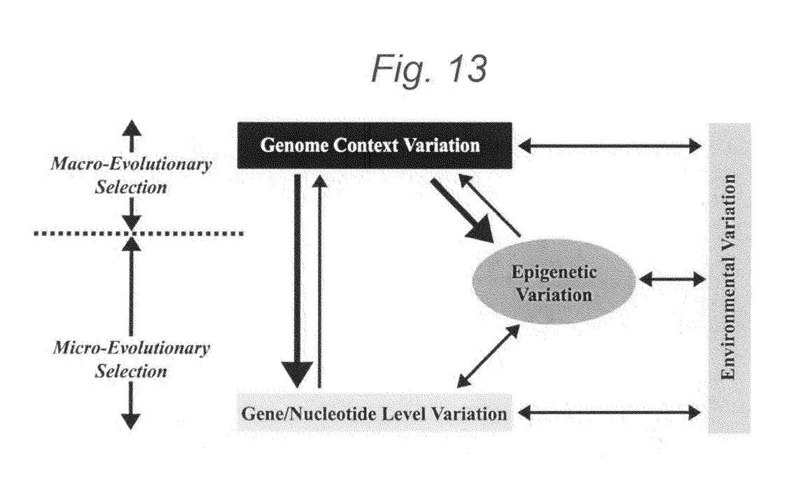

Despite the fact that epigenetic contributions to cancer have gained increasing attention in the field, their mechanism in cancer evolution is still explained within the framework of the gene mutation theory, as most studies have been designed to try to identify epigenetic change in tumor suppressor genes or genes involving genome instability. Based on the new finding that there is a collaborative and yet conflicting relationship between genes and regulators/organizers, such as the conflict between DNA sequences and chromosomal location, gene expression status and inserted genes, the understanding of the overall contribution of epigenetic regulation should not focus solely on tumor suppressor genes, but rather focus on system dynamics and evolve-ability. The genome context also defines the pattern of epigenetic regulation. This is exemplified by the fact that the epigenetic features are species specific phenomena and macro-evolution acts on the genome package level with a certain stochastic feature ensured by epigenetic regulation. One advantage of epigenetic regulation is the alteration of system dynamics without too much specificity that can be effectively adapted by the combination of genome context and environmental stress. While it is true that inappropriate gene silencing occurs involving tumor suppressor genes, the more profound changes are the increased overall level of systems dynamics, which contributes to epigenetic heterogeneity. This is illustrated by the fact that the DNA of cancer cells is generally hypomethylated leading to higher levels of gene expression for massive numbers of genes. It is possible that at certain stages of cancer progression, some pathways become dominant, but this process is stochastic and unpredictable as there are so many pathways that could be dominant depending on the possible combinations of genome context and environment. FIG. 13 illustrates the relationship among gene mutation, epigenetic response and genome alteration. Macro-evolutionary selection mainly functions at the genome level (different genome systems are defined by different karyotypes coupled to unique gene expression profiles). Microevolution mainly involves gene mutation and epigenetic responses that are responsible for a given system's micro-evolution or adaptation. In eukaryotic evolution, due to a fixed genome framework preserved by the sexual reproduction filter following speciation, an increased system complexity relies more on a layer of epigenetic regulation and copy number variation. This is important for micro-evolution and adaptation as environments are constantly changing while the framework of the genome is mainly stable.

b. Monitoring NCCA defined genome level heterogeneity is an effective way to study overall heterogeneity: Using karyotypic heterogeneity to measure genome level heterogeneity is a new approach to link population diversity to tumorigenicity. Compared with measuring heterogeneity both at the gene and epigenetic level, genome level measurements should have greater predictive power. This approach fits well with the genome-centric concept of cancer evolution as genes are genetic material for the system while epigenetic regulation is one layer of control responsible for genome modification, such as regulation of certain tissue specific genes. These two levels belong to the lower level components of a given genome (see, FIG. 13). Studies linking genome level heterogeneity to tumorigenicity by the inventors herein has led to the previously stated formula that illustrates the relationship between the evolutionary mechanism of cancer and all possible molecular mechanisms, specifically:

Evolutionary Mechanisms=εIndividual Molecular Mechanisms

The most effective way to monitor cancer progression is using the evolutionary mechanism approach. The evolutionary mechanism of cancer can be explained by three main components: instability imparts heterogeneity, which is acted on by natural selection. As each component can be impacted by a great number of genetic/epigenetic and environmental factors, it would be extremely challenging to trace each of these unlimited molecular mechanisms, where each NCCA defines a system with specific pathways (and NCCAs represent the heterogeneity of cancer evolution). On the other hand, it is relatively easy to monitor patterns of evolution, by measuring population diversity, and examining the dynamic relationship between NCCAs and CCAs.

Since cancer evolution is driven by macro-evolution and the genome is the platform of macro-selection, all genetic and epigenetic alteration will not have the same impact but can contribute to the adaptation of the genome package (see, FIG. 13). Therefore, gene or epigenetic heterogeneity may or may not be significant enough to impact genome level evolutionary outcomes. The implication of this finding is significant, as it explains why there are so many genetic/epigenetic and environmental factors linked to cancer based on small numbers of individual patients. Yet, most of them are not shared among large patient populations.

Two conclusions are derived from this analysis: (1) it is incorrect to validate mutations using large patient populations if these mutations have low penetration within a population. If the “average sample” approach is used to compare large samples to identify common patterns, the majority of lower penetrant mutations will be washed away by the analysis, becoming statistically insignificant for a population despite their strong association to individual tumors. Clearly, it is a challenge to develop an effective means to evaluate the contributions of individual mutations in a highly heterogeneous background; and (2) the predictability of cancer can be accomplished by measuring the system heterogeneity that is shared by most patients rather than characterize each of the individual factors that contributes to cancer.

The scoring and monitoring of genome level heterogeneity is straightforward. Following chromosomal preparation from targeted cell cultures, altered karyotypes are identified by multiple color spectral karyotypes and the frequency of non-clonal chromosome aberrations are calculated among analyzed mitotic figures. However, this relatively simple approach has technical limitations for in vivo analysis, as it requires a dividing cell population to prepare mitotic figures. New methods, such as interphase FISH detection using multiple probes or other in vivo visualization methods might solve this limitation to a certain degree. Single cell based array CGH could also be useful. A major challenge is the fact that there are many subtypes of structural NCCAs, including DMFs, chromosome fragmentation, and numerical NCCAs including polyploidy and aneuploidy, plus copy number variations at the single cell level, which requires a combination of SKY and single cell CGH analysis. Clearly, a methodological breakthrough is needed to effectively monitor genome level heterogeneity, and to quantitatively measure heterogeneity from different levels, and to predict the probability of cancer evolution in terms of estimating the speed and likelihood of progression. This knowledge will help us to understand the conflicting and collaborative relationships among key genetic and epigenetic levels, to establish the adaptive landscape model based on different levels of heterogeneity, and to apply them to clinical situations.

FIG. 13 illustrates the interactive relationship between multiple levels of heterogeneity and types of evolutionary selection. It is seen from this figure that despite the close interaction among gene, epigenetic, and genome levels, it is genome level heterogeneity that is primarily responsible for cancer evolution.

As system instability is best monitored at the genome level rather than the gene or epigenetic level, and the determination of frequencies of NCCAs is the best method of measuring reliably the level of genome system instability, it is logical to implement this concept into the clinical setting. In light of the data that links elevated NCCAs to disease conditions (such as prostate cancer data and Gulf War Illness data), there is now an urgent need to carry out large scale clinical testing to evaluate the clinical usefulness of NCCAs.

Data that Demonstrates that Increased NCCAs Represent the General System's Behavior when a System is Under Stress (Both Internal and Environmental Stresses)

The general strategy of studying bio-molecular mechanisms relies on the establishment of a causative relationship following specific experimental manipulation such as molecular targeting. This approach not only represents the gold standard in molecular research, but also provides the basis for many molecular medical interventions, including gene therapy and target specific drug therapy. However, this well accepted approach is now being challenged, as an increasing number of pathways are being identified following a specific targeting event, and by dynamic and unpredictable (and even contradictory) interactions occurring among pathways and among cells and between different time windows of observation. Within a complex system, much of the designed specific targeting does not lead consistently to a specific response, as a certain degree of stochastic change is an integral feature of complex systems, and the true function and importance of a specific gene is only relevant to a given genome within a given environment. To further illustrate the relationship between a system's response and specific molecular targeting events, one needs to examine whether molecular targeting can lead to overall system changes when the selected pathways are altered experimentally.

The experimental criterion that determines a changing genome system according to the genome theory herein established, the genome context (defined as the total gene content plus their genomic topology), refers to an entire set of genes/regulatory elements and their physical distribution along a chromosome and the topological interaction between chromatin domains within the nucleus, and it is these relationships that determine the genetic system. The genome theory is based on the idea that given a certain level of complexity, it is the genome package rather than specific genes that define a genetic network; and when there is a large number of chromatin constrained genes, these genetic agents can form complex networks according to the self-organization principle. Therefore, karyotypic changes can be used as a criterion to judge whether the genome system is altered.

In order to examine directly the relationship between specific molecular targeting and genome system evolution, various types of experimental systems have been used for molecular targeting followed by karyotype and pathway analysis. The targeting events used include both highly specific and less specific molecular interventions focusing on a specific gene or pathway, or inducing both general and more specific environmental stresses. Surprisingly, in most of the systems examined, the targeted systems became altered as evident by increased genome level dynamics and the formation of new karyotypes in a stochastic fashion. Furthermore, a common pattern that emerged from this diverse study was the shared relationship between the system stress (achieved by various molecular targeting events), elevated genome level dynamics (reflected by the elevated frequencies of non-clonal chromosome aberrations or NCCAs) and stochastic genome evolution (marked by the new formed karyoypes). Regardless of how specific the initial molecular targeting is, the system responds with an increase in instability, which leads to stochastic system evolution when system recovery is no longer an option.

Experimental Results

There are many ways to target specific parts of the genetic system. To date, however, there has been very little follow-up to determine the extent of any genome level alteration that might be caused by targeting experiments, as the causative effects often are studied immediately following the molecular targeting without tracing the long term effects. To examine whether specific molecular targeting can lead to genome evolution or macro-evolution as reflected by newly formed karyotypes, the inventors have elected to study both the short and long term effects, and have selected some typical model systems that have been widely used in molecular biology to study specific functions of genes or pathways and the effect that alteration of these genes or pathways has on the system as a whole.

1. Expression of a specific gene that causes karyotypic changes: The over-expression of one specific gene to study its function or its impact on a given pathway or phenotype is a common practice in cancer research. Using a pair of cell lines (D2F2 and D2F2/neu) the inventors herein studied the expression of the neu gene to determine if this leads to karyotypic changes. SKY analyses were performed for this pair of cell lines. When comparing D2F2/neu with D2F2 cells, they shared one clonal chromosome aberration or CCA at t(11;16) and chromosome fragments generated from chromosomes X, 13, 3, and 1, verifying the common origin for these cells. However, most of the marker chromosomes originally detected from D2F2 were lost and a new marker chromosome was formed. In addition, transfected D2F2/neu cells exhibited a more stable karyotype as demonstrated by lower frequencies of non-clonal chromosome aberrations or NCCAs, suggesting that the process of generating the transfected cell line was selected on the basis of a less aberrant variant which expressed neu at a high level. Obviously, the experimental process of introducing neu into D2F2 cells followed by selection has triggered genome system changes through somatic cell evolution.

Additional in vitro models with over-expression of specific genes have been examined and they commonly display increased genome dynamics and many of them also display karyotypic changes compared to control lines, including HoxA1, Rad6, Cyckin D1 and HPV16 E6. Similarly, transgenic mice carrying over expressed “cancer genes” often displaying increased genome level dynamics both in in vitro and in vivo models.

2. Knockout of a specific gene using siRNA techniques lead to karyotypic changes: SiRNA technology is a powerful method of studying functions of individual genes. It would be interesting to investigate if this procedure could lead to changes in the genome system as well. Rad6B, a fundamental component of the postreplication DNA repair pathway, was used for this testing.

SKY data illustrated that the karyotypes were altered. A number of additional SiRNA constructs were examined and all shared an altered genome as illustrated by SKY analysis, suggesting that gene targeting by siRNA often leads to system evolution. The new information here is, that the relationship between a given gene target and increased system dynamics (indicated by elevated NCCAs) might not be that specific, as many gene targeting events can lead to altered systems as reflected by initially elevated NCCAs and then the newly formed stable karyotypes.

3. Less specific targeted drugs can cause karyotypic changes: A well accepted mechanism of drug resistance acquisition is a specific gene mutation linked to a defined molecular pathway such as the cell death pathway, multiple drug resistance pathway or chromosomal machinery that is responsible for genome integrity. In order to test such a mechanism in the context of genome system evolution, well characterized ovarian cancer cell lines were treated with cisplatin, which is a drug commonly used to treat various types of cancers. The mechanism of this drug is thought to be binding and cross-linking of DNA by platinum complexes, which ultimately triggers apoptosis. The mechanisms of cisplatin resistance have been proposed to include changes in cellular uptake, increased detoxification of the drug, inhibition of apoptosis and increased DNA repair.

Following 6-9 months of selection by gradually increased concentrations of cisplatin, a number of stable drug resistant clones were independently selected from OV433 and TOV112D cell lines. The karyotypes were examined by SKY and some key pathways possibly related to the acquisition of drug resistance in the clones were analyzed by western blots.

Despite the fact that all selected clones displayed stable drug resistant phenotypes at high dosages, they all had drastically different karyotypes. According to the genome theory, different karyotypes represent different genome systems as the genome context defines the system by providing a specific genetic network. Among the many different formed genomes that displayed drug resistance, one would expect also to detect diverse pathways associated with these different genomes as each new formed system often adopts different pathways. The molecular mechanisms of cisplatin resistance were analyzed in the two resistant lines to illustrate this point. Indeed, they displayed different molecular pathways likely associated with the drug resistance. While the increased MKP-1 protein level and decreased Bcl-2 and Bim protein level was observed from cisplatin resistant clone OV433 cells, increased Bcl-2 and Bim expression and similar MKP-1 expression levels were detected from resistant clone TOV112D cells.

4. Drugs with specific targets that cause karyotypic change: It has been suggested that target specific treatments will have a specific effect due to their molecular causative relationship. To examine this issue, TW-37, a small-molecule inhibitor drug that specifically binds to and inhibits Bcl-2 was used in a cisplatin resistance cell line that was previously tested. Under treatment conditions resulting in 50% cell death, surviving cells of two cell lines were followed with SKY analysis. The frequencies of NCCAs were increased indicating increased instability of the tested cell population. After a few weeks of culture, new stable karyotypes were detected.

This result shows that the specific small-molecule inhibitors also can generate system instability leading to genome system evolution and not just simply the specific and limited effects of inhibiting the Bcl-2 target. The observed elevated genome instability following specific molecular targeting is significant, and calls for the re-examination of the issue of molecular causative relationships from a system point of view.

5. A comparison of specific and non-specific drugs that target the same pathway: In order to compare directly the effects of challenging the same molecular pathway with specific or nonspecific drug treatments with regard to genome system stability and evolution, the protein degradation pathway was chosen. Two proteasome inhibitors, Bortezomib (also named Velcade or PS-341) and MG132 were used. Bortezomib is a boronic peptide that displays high specificity toward the b5 subunit of the proteasome. MG132 is a peptide aldehyde, which can target both serine/threonine and cysteine proteases due to the highly active aldehyde group. Compared to 132, Bortezomib is more specific. Two breast cancer cell lines, MDA-MB-231 and BT549, were used for this comparison. Immediately following treatment of cell line MDA-MB-231, populations treated by both drugs showed an increase in NCCAs. The elevated frequencies of NCCAs were similar between the two drugs, indicating that there is an increase in population diversity regardless of the specificity of the treatment. In contrast, in cell line BT549, both drugs failed to generate increased NCCA frequencies, demonstrating a difference among different genome systems. Interestingly, in both positive and negative responses, the more specific and less specific inhibitors seem to induce a similar response by the system. After a few weeks culture, there seemed to be no drastic changes in terms of the formation of new karyotypes.

6. A comparison of low and high stresses and their impact on genome evolution: Taken together, the above diverse experiments suggest that it is not an issue of the specific or nonspecific nature of experimental manipulations that matters most with regard to system dynamics and evolution. By comparing various treatments and their consequences (elevated NCCAs), it was realized that the common link between the types and levels of stress is induction of system dynamics by the experiments as illustrated by the link between the frequencies of NCCAs and system stress. This agrees with the newly established relationship between the evolutionary mechanism and individual molecular mechanisms, where the stress is the general cause for the system dynamics and heterogeneity. Stress is the common denominator between diverse molecular pathways and cancer evolution. Since any experimental manipulation represents a stress, it is thus possible that the key to system evolution is the level of stress the system faces. The specificity of the stress is less important than inducing system evolution, as stress represents a secondary factor within a complex system.

To illustrate this issue further, the inventors herein have carried out the following experiments that represent different types of stress that the system faces. All of the stress introduced should induce instability and promote genome evolution in vitro.

First, the same cell line was used with both “high” and “low” levels of stress applied respectively. High stress was applied through chemo-therapy drug treatments (doxorubicin) (Dox) while low stress was generated by altering culture conditions. During drug treatment, a high level of NCCAs was quickly detected immediately after treatment of a high dosage, and the cell population kept its high dynamic/diversity status even after withdrawal of the drugs. This was illustrated by the drastically altered karyotypes in every cell examined, which persisted until certain karyotypes became dominant (completing the transition from high levels of NCCAs to specific CCAs) following at least 6 weeks of culture in a drug free medium.

Variable cell culture conditions were tested including the altered concentration of serum in culture medium and changes to the temperature of cell cultures, aimed at mimicking different stresses. In contrast to Dox treated cells, in cells subjected to a less than 1% serum culture there was no immediate significant increase in NCCAs, indicating that the 1% serum did not induce a high level of stress on the cell population. However, following six months of continuous culture in 1% serum, a new dominant karyotype formed with a specific marker chromosome. The degree of karyotypic change was minimal when compared to the Dox treated group.

Increased culture temperature was also applied to a culture in 10% serum. 24 hours following the temperature increase (37° C. to 42° C.), increased NCCAs were detected. The elevated NCCAs can be detected for a number of days, and then, the NCCAs recede back to a normal level. When the culture is switched from 42° C. to 37° C., the elevated NCCAs were again observed, and at a surprisingly higher level compared with the initial switch from 37° C. to 42° C., indicating that the normal temperature now becomes a new stress for the already adapted cell population (at 42° C.).

The inventors herein then determined to use a more defined stress condition to validate the link between the stress and the genome evolution. Since the endoplasmic reticulum (ER) has an important role in sensing cellular stress, and the ER stress is well studied, the inventors hypothesize that the defined ER stress will also generate elevated NCCAs leading to the formation of new karyotypes. Specific agents were used to target specific pathways of ER stress including tharpsigargin, tunicamycin and DTT. Tunicamycin interferes with glycalation and results in induction of ER stress. Tharpsigargin inhibits the endoplasmic reticulum calcium ATPase, and results in the release of calcium from the ER, and DTT induces reductive stress through its activity in breaking disulfide bonds which leads to induction of the unfolded protein response. To show that the ER response pathway is active following the drug treatment western blot analysis was carried out. As illustrated, the specific ER stress pathways were indeed active in three treatments. SKY analysis then was used to examine the karyotypes and the NCCA levels of the treated cells, and elevated NCCAs were observed for all three treatments following a short period of culture.

Together these experiments illustrate that the key link is the stress and the system dynamics reflected as the NCCA levels. The stress is important to system evolution regardless of the type of stress that is applied. Under high stress, the system undergoes drastic changes and the speed of genome evolution seems much more rapid and the degree of genome level changes is more profound. Under low stress, the system undergoes fewer changes and it takes a much longer time for the formation of new karyotypes and does so with a lower degree of overall alteration

Although the invention has been described in terms of specific embodiments and applications, persons skilled in the art may, in light of this teaching, generate additional embodiments without exceeding the scope or departing from the spirit of the claimed invention. Accordingly, it is to be understood that the drawing and description in this disclosure are proffered to facilitate comprehension of the invention, and should not be construed to limit the scope thereof.

What is claimed is:

1. A diagnostic method of determining tumorigenicity of a tissue specimen, the method comprising the steps of:
determining a magnitude of genome diversity in the tissue specimen; and
diagnosing a likelihood of cancer in response to said step of determining the magnitude of genome diversity.

2. The diagnostic method of claim 1, wherein said step of determining the magnitude of genome diversity comprises the step of determining the karyotypic heterogeneity in the tissue specimen.

3. The diagnostic method of claim 1, wherein said step of determining the presence of elevated genome diversity comprises the step of detecting non-clonal chromosome aberrations (NCCAs).

4. The diagnostic method of claim 3, wherein said step of detecting NCCAs comprises the further step of detecting the frequency of NCCAs.

5. The diagnostic method of claim 4, wherein said step of diagnosing is responsive to said step of detecting the frequency of NCCAs.

6. The diagnostic method of claim 3, wherein said step of detecting NCCAs comprises the further step of screening lymphocytes.

7. The diagnostic method of claim 1, wherein said step of determining the presence of elevated genome diversity comprises the step of applying Spectral Karyotyping to detect translocations throughout the genome.

8. A diagnostic method of determining drug resistance of a patient, the method comprising the steps of:
determining the presence of genome diversity in the tissue specimen; and
diagnosing the drug resistance of the patient in response to said step of determining the presence of genome diversity.

9. A method of determining the effectiveness of a drug as well as determining the potential damage of genome instability, the method comprising the steps of:
evaluating the effectiveness of inducing DMFs or chromosome fragmentation; and
evaluating the induced frequency of NCCAs.

10. A method of measuring an overall system stress level, the method comprising the step of using the frequencies of NCCAs to measure stress.

11. A method of identifying populations with unstable genomes, the method comprising the step of determining the level of NCCAs in blood cell cultures.

12. A method of diagnosing a complex disease, the method comprising the step of determining whether genome instability is the main contributing factor.

13. A method of determining the potential side effects of drugs, the method comprising the step of determining the induced levels of NCCAs in cultured cell lines with defined genome instability.

14. A method of estimating the stress level for individuals exposed to chemical or other types of stress, the method comprising the step of determining from patient specimen the induced level of NCCAs.

15. The method of claim 14, wherein the patient specimen is a blood specimen.

16. The method of claim 14, wherein the patient specimen is a specific tissue specimen.
